(19) United States
(12) Patent Application Publication
(10) Pub. No.: US 2003/0220374 A1
(43) Pub. Date: Nov. 27, 2003

(54) Compositions and methods of treatment involving peroxisome proliferator-activated receptor-gamma agonists and cyclooxygenase-2 selective inhibitors

(75) Inventor: Philip Needleman, Creve Coeur, MO (US)

Correspondence Address: Charles E. Dunlap, Keenan Building, Third Floor, 1330 Lady Street, Columbia, SC, 29201, US

(73) Assignee: Pharmacia Corporation, St. Louis, MO

(21) Appl. No.: 10/341,174

(22) Filed: Jan. 13, 2003

Related U.S. Application Data
(60) Provisional application No. 60/348,298, filed on Jan. 14, 2002.

(51) Int. Cl. A61K 31/4439; A61K 31/426; A61K 31/405; A61K 31/192; A61K 31/557

(52) U.S. Cl. 514/342; 514/369; 514/420; 514/570; 514/573; 514/569

(57) ABSTRACT

Methods for the treatment, prevention, or inhibition of pain, inflammation, or inflammation-related disorder, and for the treatment or inhibition of cardiovascular disease or disorder, and for the treatment or inhibition of cancer in a subject in need of such treatment, prevention, or inhibition, include treating the subject with a peroxisome proliferator activated receptor-γ agonist and a cyclooxygenase-2 selective inhibitor or prodrug thereof. Compositions, pharmaceutical compositions and kits for effecting the particular methods are also described.

### CROSS-REFERENCE TO RELATED APPLICATIONS

This application is related to, and claims priority to, U.S. Provisional Patent Application Serial No. 60/348,298, filed Jan. 14, 2002, which is hereby incorporated by reference herein in its entirety.

### BACKGROUND OF THE INVENTION

(1) Field of the Invention

The present invention relates to compositions that include peroxisome proliferator-activated receptor agonists and cyclooxygenase-2 selective inhibitors, and more particularly to compositions that include peroxisome proliferator-activated receptor gamma agonists and cyclooxygenase-2 selective inhibitors and their use for the treatment, prevention, or inhibition of cancer, cardiovascular disease or disorder, Alzheimer's disease, and pain, inflammation, or inflammation-related disorder.

(2) Description of the Related Art

Peroxisome proliferator-activated receptors (PPARs) belong to the nuclear receptor superfamily of ligand-activated transcription factors. Once bound by a ligand, PPARs heterodimerize with 9-cis retinoic acid receptors (RXRs) in the nucleus. These heterodimers bind to specific peroxisome-proliferator response elements (PPRE) in the promoter of target genes, thereby regulating transcription and expression of these genes. Three isoforms of PPARs, alpha, delta, and gamma, have been identified and differ in their tissue distribution, affinity for particular ligands, and physiological consequences. See, e.g., Corton, J. C. et al., Annu. Rev. Pharmacol. Toxicol., 40:491-518 (2000), and Chawla, A. et al., Science, 294:1866-1870 (2001).

Of particular importance is PPAR gamma (PPARγ), which is activated by binding with such compounds as thiazolidinediones and prostaglandin J₂ and its analogs. Activation of PPARγ by ligand binding results in changes in the expression of genes important in glucose and lipid metabolism. See, e.g., Olefsky, J. M. and Saltiel, A. R., Trends Endocrinol. Metab., 11(9):362-368 (2000); and Koomers, R. and Vrana, A., Physiol. Res., 47:215-225 (1998).

As a consequence of these changes in gene expression, thiazolidinediones, or “glitazones,” function as insulin-sensitizers, and they have been successfully used for the treatment of type 2 diabetes. Moreover, thiazolidinediones reduce circulating free fatty acids and decrease triglyceride levels, an additional therapeutic benefit to patients with type 2 diabetes who often suffer from high cholesterol levels. Roth, D. L. and Zick, Y., Diabetes Care, 24(3):588-597 (2001).

Ligands that cause some physiological consequence by binding with a receptor can be referred to as agonists. Emerging evidence indicates that PPARγ agonists have potential clinical uses beyond treatment of type 2 diabetes. PPARs modulate the inflammatory response, and PPARγ agonists have been shown to exert anti-inflammatory effects by inhibiting the expression of pro-inflammatory genes such as cytokines, metalloproteases, and acute-phase response genes. See, e.g., Delerive, P. et al., J. Endocrinol., 169(3):453-459 (2001); Gelman, L. et al., Cell. Mol. Life. Sci., 55:932-943 (1999); and U.S. Pat. No. 5,925,657. Rheumatoid arthritis and other inflammatory conditions are characterized by increased expression of these proteins.

It is also believed that activation of PPARγ modulates the expression of cyclooxygenase-2 (Cox-2). Cox-2, along with the constitutive Cox-1 enzyme, catalyzes an initial step in the synthesis of prostaglandins, which are known mediators of inflammation. Combs, C. K. et al., J. Neurosci., 20(2):558-567 (2000); and U.S. Pat. No. 6,191,154. Cox-2-selective inhibitors, which are discussed in more detail below, are particularly useful in the treatment of pain and inflammation because they inhibit prostaglandin production while leaving the beneficial activities of Cox-1 intact. Ikawa et al., in Exp. Cell Res., 267(1):73-80 (2001), reported that PPARγ stimulated Cox-2 expression by up-regulation of the TNFα pathway. These findings indicate a potential therapeutic application for PPARγ agonists in the treatment of a variety of inflammatory diseases.

PPARγ agonists also exert effects on the cardiovascular system. See, e.g., Marx, N. et al., J. Cardiovasc. Risk, 8:203-210 (2001). As noted above, thiazolidinediones reduce circulating free fatty acids and triglyceride levels. Additionally, a slight increase in HDL levels, i.e., the “good” cholesterol, was observed in patients treated with the thiazolidinedione pioglitazone. See, e.g. http://www.diabetesnet.com/glitazones.html. Thus, these alterations in lipids could help reduce or prevent cardiac events in patients.

An increasing body of evidence supports the hypothesis that atherosclerosis shares many similarities with inflammatory diseases. Neve, B. P. et al., Biochem. Pharmacol., 60:1245-1250 (2000). In particular, atherosclerosis has been shown to have an inflammatory profile similar to that of rheumatoid arthritis. Pasceri V. et al., Circulation, 100:2124-2126. Atherosclerosis is characterized by the occurrence of lesions that may result in ischemia of the heart, brain, or extremities, thereby leading to infarction. The formation of atherosclerotic lesions involves the attraction of monocytes/macrophages and T lymphocytes to the blood vessel wall and the migration and proliferation of vascular smooth muscle cells, resulting in narrowing of the vessel lumen. See, e.g., Neve, B. P. et al., Biochem. Pharmacol., 60:1245-1250 (2000). PPARγ ligands inhibit the production of inflammatory cytokines by activated monocytes and decrease the transcription of monocyte chemoattractant protein (MCP-1). Jiang, C. et al., Nature, 391:82-86 (1998); and Murao, K. et al., FEBS Lett., 454:27-30 (1999). Recent research further suggests that PPARγ may influence monocyte recruitment and cholesterol efflux from foam cells, important events in the development of atherosclerosis. Chinetti, G. et al., Circulation, 101:2411-2417 (2000). Moreover, the PPARγ agonist troglitazone inhibits vascular smooth muscle cell growth and decreases hyperplasia of human carotid arteries. Law, R. et al., J. Clin. Invest., 98:1897-1905 (1998). Therefore, it is believed that PPARγ activators could be used clinically to inhibit the lesion formation and vessel narrowing associated with atherosclerosis and other cardiovascular diseases.

PPARγ has also been implicated in vitro in the regulation of growth and differentiation of human cancer cells, thereby supporting a therapeutic role for PPARγ agonists in the prevention or treatment of cancer. See, e.g., Corton, J. C. et al., Annu. Rev. Pharmacol. Toxicol., 40:491-518 (2000); and Gelman, L. et al., Cell. Mol. Life. Sci., 55:932-943 (1999). The thiazolidinediones, troglitazone and pioglitazone, as well as the endogenous PPARγ ligand 15-deoxy-Δ^{12,14}-prostaglandin J₂, caused marked growth inhibition of hepatocellular cancer cells, and troglitazone inhibited the growth of human lung cancer cells through the induction of apoptosis. Rumi, M. A. et al., Br. J. Cancer, 84(12):1640-1647 (2001); and Tsubouchi, Y. et al., Biochem. Biophys. Res. Comm., 270(2):400-405 (2000). Similar anti-proliferative effects of PPARγ agonists have been reported in vitro with human breast, prostatic, and pancreatic cancer cells. Elnemr, A. et al., Int. J. Oncol., 17(6):1157-1164 (2000); Yee, L. D. et al., Int. J. Oncol., 15(5):967-973 (1999); and Gelman, L. et al., Cell. Mol. Life. Sci., 55:932-943 (1999). Therefore, it is believed that thiazolidinediones and other PPARγ agonists may be useful therapeutic agents in the treatment of a variety of cancers.

Uryu, S. et al., in Brain Res. 924(2):229-236(2002) have proposed that PPAR-gamma agonists, such as troglitazone, may provide a novel therapy for various neurodegenerative diseases, such as Alzheimer's disease.

The potential uses of PPARγ agonists for the treatment of insulin resistance and obesity has been discussed by Schwartz, M. W. et al., Nature, 402:860-861 (1999).

As discussed briefly above, compounds that selectively inhibit the cyclooxygenase-2 enzyme have been discovered. These compounds selectively inhibit the activity of Cox-2 to a greater extent than the activity of Cox-1. The new Cox-2-selective inhibitors are believed to offer advantages that include the capacity to prevent or reduce inflammation while avoiding harmful side effects associated with the inhibition of Cox-1. Thus, cyclooxygenase-2-selective inhibitors have shown great promise for use in therapies—especially in therapies that require extended administration, such as for pain and inflammation control for arthritis. Additional information on the identification of cyclooxygenase-2-selective inhibitors can be found in: (1) Buttgereit, F. et al., Am. J. Med., 110(3 Suppl. 1):13-9 (2001); (2) Osiri, M. et al., Arthritis Care Res., 12(5):351-62 (1999); (3) Buttar, N. S. et al., Mayo Clin. Proc., 75(10):1027-38 (2000); (4) Wollheim, F. A., Current Opin. Rheumatol., 13:193-201 (2001); (5) U.S. Pat. Nos. 5,434,178 (1,3,5-trisubstituted pyrazole compounds); (6) 5,476,944 (derivatives of cyclic phenolic thioethers); (7) 5,643,933 (substituted sulfonylphenylheterocycles); 5,859,257 (isoxazole compounds); (8) 5,932,598 (prodrugs of benzenesulfonamide-containing Cox-2 inhibitors); (9) 6,156,781 (substituted pyrazolyl benzenesulfonamides); and (10) 6,110,960 (for dihydrobenzopyran and related compounds).

The efficacy and side effects of cyclooxygenase-2-selective inhibitors for the treatment of inflammation have been reported. References include: Hillson, J. L. et al., Expert Opin. Pharmacother., 1(5):1053-66 (2000), (for rofecoxib, Vioxx®, Merck & Co., Inc.); Everts, B. et al., Clin. Rheumatol., 19(5):331-43 (2000), (for celecoxib, Celebrex®, Pharmacia Corporation, and rofecoxib); Jamali, F., J. Pharm. Pharm. Sci., 4(1):1-6 (2001), (for celecoxib); U.S. Pat. Nos. 5,521,207 and 5,760,068 (for substituted pyrazolyl benzenesulfonamides); Davies, N. M. et al., Clinical Genetics, Abstr. at http://www.mmhc.com/cg/articles/CG0006/davies.html (for meloxicam, celecoxib, valdecoxib, parecoxib, deracoxib, and rofecoxib); http://www.celebrex.com (for celecoxib); http://www.docguide.com/dg.nsf/PrintPrint/F1F8DDD2D8B0094085256 98F00742187, May 9, 2001 (for etoricoxib, MK-663, Merck & Co., Inc.); Saag, K. et al., Arch. Fam. Med., 9(10):1124-34 (2000), (for rofecoxib); International Patent Publication No. WO 00/24719 (for ABT 963, Abbott Laboratories).

Cox-2 inhibitors have also been described for the treatment of cancer (WO98/16227) and for the treatment of tumors (See, EP 927,555, and Rozic et al., Int. J. Cancer, 93(4):497-506 (2001)). Celecoxib®, a selective inhibitor of Cox-2, exerted a potent inhibition of fibroblast growth factor-induced corneal angiogenesis in rats. (Masferrer et al., Proc. Am. Assoc. Cancer Research 1999, 40: 396). WO 98/41511 describes 5-(4-sulphunyl-phenyl)-pyridazinone derivatives used for treating cancer. WO 98/41516 describes (methylsulphonyl)phenyl-2-(5H)-furanone derivatives that can be used in the treatment of cancer. Kalgutkar, A. S. et al., Curr. Drug Targets, 2(1):79-106 (2001) suggest that Cox-2 selective inhibitors could be used to prevent or treat cancer by affecting tumor viability, growth, and metastasis. Masferrer et al., in Ann. NY Acad. Sci., 889:84 -86 (1999) describe Cox-2 selective inhibitors as antiangiogenic agents with potential therapeutic utility in several types of cancers. The utility of Cox-2 inhibition in clinical cancer prevention was described by Lynch, P. M., in Oncology, 15(3):21-26 (2001), and Watanabe et al., in Biofactors 2000, 12(1-4):129-133 (2000) described the potential of Cox-2 selective inhibitors for chemopreventive agents against colon cancer.

Additionally, various combination therapies using Cox-2 inhibitors with other selected combination regimens for the treatment of cancer has also been reported. See e.g., FR 27 71 005 (compositions containing a cyclooxygenase-2 inhibitor and N-methyl-d-aspartate (NMDA) antagonist used to treat cancer and other diseases); WO 99/18960 (combination comprising a cyclooxygenase-2 inhibitor and an induced nitric-oxide synthase inhibitor (iNOS) that can be used to treat colorectal and breast cancer); WO 99/13799 (combination of a cyclooxygenase-2 inhibitor and an opioid analgesic); WO 97/36497 (combination comprising a cyclooxygenase-2 inhibitor and a 5-lipoxygenase inhibitor useful in treating cancer); WO 97/29776 (composition comprising a cyclooxygenase-2 inhibitor in combination with a leukotriene B4 receptor antagonist and an immunosuppressive drug); WO 97/29775 (use of a cyclooxygenase-2 inhibitor in combination with a leukotriene A4 hydrolase inhibitor and an immunosuppressive drug); WO 97/29774 (combination of a cyclooxygenase-2 inhibitor and protstagladin or antiulcer agent useful in treating cancer); WO 97/11701 (combination comprising of a cyclooxygenase-2 inhibitor and a leukotriene B receptor antagonist useful in treating colorectal cancer); WO 96/41645 (combination comprising a cyclooxygenase-2 inhibitor and leukotriene A hydrolase inhibitor); WO 96/03385 (3,4,-Di substituted pyrazole compounds given alone or in combination with NSAIDs, steroids, 5-LO inhibitors, LTB4 antagonists, or LTA4 hydrolase inhibitors for the treatment of cancer); WO 98/47890 (substituted benzopyran derivatives that may be used alone or in combination with other active principles); WO 00/38730 (method of using cyclooxygenase-2 inhibitor and one or more antineoplastic agents as a combination therapy in the treatment of neoplasia); Mann, M. et al., Gastroenterology, 120(7):1713-1719 (2001) (combination treatment with Cox-2 and HER-2/neu inhibitors reduced colorectal carcinoma growth).

Other reports have indicated the Cox-2 selective inhibitors have cardiovascular applications. For example, Saito, T. et al., in Biochem. Biophys. Res. Comm., 273:772-775 (2000), reported that the inhibition of Cox-2 improves cardiac function in myocardial infarction. Ridker, P. M. et al., in The New England J. of Med., 336(14):973-979 (1997), raised the possibility that anti-inflammatory agents may have clinical benefits in preventing cardiovascular disease. In addition, Cox-2 selective inhibitors have been proposed for therapeutic use in cardiovascular disease when combined with modulation of inducible nitric oxide synthase (See, Baker, C. S. R. et al., Arterioscler. Thromb. Vasc. Biol., 19:646-655 (1999)), and with HMG-COA reductase inhibitor (U.S. Pat. No. 6,245,797).

It would be useful, therefore, to provide an effective method for the treatment, prevention, or inhibition or pain, inflammation, or inflammation-related disorder, and also an effective method for the treatment and prevention of cancer and cardiovascular disease or disorder. It would also be useful if these methods provided beneficial properties that were not provided by known and conventional methods of treatment for these conditions.

### SUMMARY OF THE INVENTION

Briefly, therefore the present invention is directed to a novel method for the prevention, treatment, or inhibition of pain, inflammation, or inflammation-related disorder, or cancer, or Alzheimer's disease, or cardiovascular disease or disorder in a subject in need of such treatment, prevention, or inhibition, the method comprising treating the subject with a peroxisome proliferator activated receptor-7 agonist and a cyclooxygenase-2 selective inhibitor or prodrug thereof.

The present invention is also directed to a novel method for the treatment or prevention of disorders having an inflammatory component in a subject in need of the treatment or prevention of disorders having an inflammatory component, the method comprising administering to the subject a therapeutically effective dose of a peroxisome proliferator activated receptor-γ agonist and a cyclooxygenase-2 selective inhibitor or a pharmaceutically acceptable salt or prodrug thereof.

The present invention is also directed to a novel composition for the treatment, prevention, or inhibition or pain, inflammation, or inflammation-associated disorder comprising a peroxisome proliferator activated receptor-γ agonist and a cyclooxygenase-2 selective inhibitor or prodrug thereof.

The present invention is also directed to a novel pharmaceutical composition comprising a peroxisome proliferator activated receptor-γ agonist; a cyclooxygenase-2 selective inhibitor or prodrug thereof; and a pharmaceutically-acceptable excipient.

The present invention is also directed to a novel kit that is suitable for use in the treatment, prevention or inhibition of pain, inflammation or inflammation-associated disorder, the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the compounds for the treatment, prevention, or inhibition of pain, inflammation or inflammation-associated disorder.

The present invention is also directed to a novel method for the treatment, prevention, or inhibition of cardiovascular disease or disorder in a subject in need of such treatment, prevention, or inhibition, the method comprising treating the subject with a peroxisome proliferator-activated receptor-γ agonist and a cyclooxygenase-2 selective inhibitor or a pharmaceutically acceptable salt or prodrug thereof.

The present invention is also directed to a novel composition for the treatment, prevention, or inhibition of cardiovascular disease or disorder comprising a peroxisome proliferator activated receptor-γ agonist and a cyclooxygenase-2 selective inhibitor or prodrug thereof.

The present invention is also directed to a novel kit that is suitable for use in the treatment, prevention, or inhibition of cardiovascular disease or disorder, the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the compounds for the treatment, prevention, or inhibition of cardiovascular disease or disorder.

The present invention is also directed to a novel method for the treatment, prevention, or inhibition of cancer in a subject in need of such treatment, prevention, or inhibition, the method comprising treating the subject with a peroxisome proliferator-activated receptor-γ agonist and a cyclooxygenase-2 selective inhibitor or a pharmaceutically acceptable salt or prodrug thereof.

The present invention is also directed to a novel composition for the treatment, prevention, or inhibition of cancer comprising a peroxisome proliferator activated receptor-γ agonist and a cyclooxygenase-2 selective inhibitor or prodrug thereof.

The present invention is also directed to a novel kit that is suitable for use in the treatment, prevention, or inhibition of cancer, the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the compounds for the treatment, prevention, or inhibition of cancer.

The present invention is also directed to a novel method for the treatment, prevention, or inhibition of Alzheimer's disease in a subject in need of such treatment, prevention, or inhibition, the method comprising treating the subject with a peroxisome proliferator-activated receptor-γ agonist and a cyclooxygenase-2 selective inhibitor or a pharmaceutically acceptable salt or prodrug thereof.

The present invention is also directed to a novel composition for the treatment, prevention, or inhibition of Alzheimer's disease comprising a peroxisome proliferator activated receptor-γ agonist and a cyclooxygenase-2 selective inhibitor or prodrug thereof.

The present invention is also directed to a novel kit that is suitable for use in the treatment, prevention, or inhibition of Alzheimer's disease, the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the compounds for the treatment, prevention, or inhibition of Alzheimer's disease.

Among the several advantages found to be achieved by the present invention, therefore, may be noted the provision of an effective method for the treatment, prevention, or inhibition or pain, inflammation, or inflammation-related disorder, and also an effective method for the treatment and prevention of cancer, Alzheimer's disease, and cardiovascular disease or disorder, the provision of such methods that provided beneficial properties that are comparable to or superior to those provided by known and conventional methods of treatment for these conditions, and the provision of compositions, pharmaceutical compositions and kits to effect these methods.

### DETAILED DESCRIPTION OF THE PREFERRED EMBODIMENTS

In accordance with the present invention, it has been discovered that pain, inflammation and inflammation-associated disorders, as well as cardiovascular diseases and disorders, Alzheimer's disease, and cancer can be effectively prevented, inhibited, and/or treated in subjects that are in need of such prevention, inhibition, or treatment by treating the subject with a combination that includes a peroxisome proliferator-activated receptor-gamma (PPARγ) agonist and one or more cyclooxygenase-2 selective inhibitors.

The amount of the PPARγ agonist and the amount of the cyclooxygenase-2-selective inhibitor that are used in the treatment can be selected so that together they constitute a pain or inflammation suppressing treatment or prevention effective amount, or a cardiovascular disease or disorder treatment or prevention effective amount, or an Alzheimer's disease treatment or prevention effective amount, or a cancer treatment or prevention effective amount.

The novel method of treating a subject with a combination of a PPARγ agonist and a cyclooxygenase-2-selective inhibitor provides a safe and efficacious method for preventing and alleviating pain and inflammation and for preventing and treating disorders that are associated with inflammation, as well as for treating and prevention cardiovascular diseases and disorders, Alzheimer's disease, and cancer. In addition to being an efficacious method and composition for preventing and/or alleviating such diseases and disorders in a treated subject, such method and composition can also provide desirable properties such as stability, ease of handling, ease of compounding, reduced or lack of side effects, ease of preparation or administration, and the like.

The novel method and compositions comprise the use of a PPARγ agonist and a cyclooxygenase-2 selective inhibitor in combination.

As used herein, the terms “peroxisome proliferator activated receptor-gamma agonist”, or “PPARγ agonist” and “PPAR gamma agonist” refer to a compound or composition, which when combined with PPARγ, is capable of directly or indirectly stimulating or increasing an in vitro, ex vivo or in vivo reaction that is typical for the receptor, e.g., transcriptional regulation activity. PPARγ agonists can be identified via a variety of assays that are known to those of skill in the art, including, but not limited to, the assays described in Lehman, et al., J. Biol. Chem., 270:12953 -12956 (1995), and in U.S. Pat. Nos. 4,981,784; 5,071,773; and 6,022,897.

Preferred PPARγ agonists include thiazolidinediones (glitazones); non-steroidal anti-inflammatory drugs which are capable of binding with PPARγ—such as indomethacin, flufenamic acid, fenoprofen, and ibuprofen; unsaturated fatty acids which are capable of binding with PPARγ; prostaglandins which are capable of binding with PPARγ; and prostaglandin J₂ analogs which are capable of binding with PPARγ.

Examples of preferred PPARγ agonists are listed in Tables 1 and 2, and include, without limitation, CS-011 (CI-1037), (−)DRF2725, AD-5075, BRL49653, GW1929, AY-31367, MCC-555, JTT501, PD72953, WAY-120,744, L-764406, G1262570X (GG570), indomethacin, ciglitazone, darglitazone, englitazone, pioglitazone, rosiglitazone, troglitazone, 5-[[4-[2-(methyl-2-pyridinylamino)ethoxy]phenyl]methyl]-2,4-thiazolidinedione, docosahexaenoic acid, prostaglandin J₂, 15-deoxy-Δ^{12,14}-prostaglandin J₂, and Δ¹²-prostaglandin J₂. More preferred are glitazones, such as CS-011, AD-5075, BRL49653, AY-31637, MCC-555, ciglitazone, darglitazone, englitazone, pioglitazone, rosiglitazone, troglitazone, and 5-[[4-[2-(methyl-2-pyridinylamino)ethoxy]phenyl]methyl]-2,4-thiazolidinedione.

TABLE 1
Information about selected PPAR gamma agonists.

| | | EXPERIMENTAL | | |
|---|---|---|---|---|
| COMPOUND^{a} | CLASS | PRODUCT NO. | CAS Reg. No. | CHEMICAL NAME |
| N/A | | (−)DRF2725 | | (−)3-[4-[2-(phenoxazin-10- |
| | | | | yl)ethoxy]phenyl]=2=ethoxypropanoic acid |
| N/A | Glitazone | CS-011, CI-1037 | | |
| N/A | Glitazone | AD-5075 | | 5-[4-[2-(5-methyl-2-phenyl-4-oxazoyl)-2- |
| | | | | hydroxyethoxy]benzyl]-2,4-thiazolidinedione |
| N/A | Glitazone | BRL49653 | | 5-(4-[2-[methyl-(2- |
| | | | | pyridyl)amino]ethoxy]benzyl)thiazolidine-2,4- |
| | | | | dione |
| N/A | Tyrosine | GW1929 | | |
| | based | | | |
| N/A | Glitazone | AY-31637 | | 5-(2-naphthalenylsulfonyl)-2,4- |
| | | | | thiazolidinedione |
| N/A | Glitazone | MCC-555 | | 5-[6-(2-fluorobenzylozy)naphthalene- |
| | | | | 2ylmethyl]-2,4-thiazolidinedione |
| N/A | | JTT501 | | 4-[4-[2-(5-methyl-2-phenyl-4- |
| | | | | oxazolyl)ethoxy]benzyl]-3,5-isoxazolidinedione |
| N/A | | PD72953 | | |
| N/A | | WAY-120,744 | | |
| N/A | NSAID | L-764406 | | |
| N/A | | G1262570X, GG570 | | |
| Indomethacin | NSAID | | 53-86-1 | |
| Ciglitazone | Glitazone | | 74772-77-3 | 5-[p-[1-(methylcyclohexyl)methoxyl]benzyl]- |
| | | | | 2,4-thiazolidinedione |
| Darglitazone | Glitazone | CP-86325 | 141200-24-0 | 5-[p-[3-(5-methyl-2-phenyl-4- |
| | | | | oxazoyly)proprionyl]benzyl]-2,4- |
| | | | | thiazolidinedione |
| Englitazone | Glitazone | | 109229-58-5 | 5-[[(2R)-2-benzyl-6-chromanyl]methyl]-2,4- |
| | | | | thiazolidinedione |
| Pioglitazone | Glitazone | | 111025-46-8 | 5-[p-[2-(5-ethyl-2-pyridyl)thoxy]benzyl]-2,4- |
| (Actos ®) | | | | thiazolidinedione |
| Rosiglitazone | Glitazone | | 122320-73-4 | 5-(4-[2-(N-methyl-N-(2- |
| (Avandia ®) | | | | pyridyl)amino)ethoxy]benzyl)-2,4- |
| | | | | thiazolidinedione |
| Troglitazone | Glitazone | | 97322-87-7 | (+)-5-[[4-[(3,4-dihydro-6-hydroxy-2,5,7,8- |
| (Rezulin ®) | | | | tetramethyl-2H-1-benzopyran-2- |
| | | | | yl)methoxy]phenyl]methyl]-2,4- |
| | | | | thiazolidinedione |
| N/A | Glitazone | | | 5-[[4-[2-(methyl-2- |
| | | | | pyridinylamino)ethoxy]phenyl]methyl]-2,4- |
| | | | | thiazolidinedione |
| Docosa- | Fatty acid | | 6217-54-5 | 4,7,10,13,16,19-docosahexaenoic acid |
| hexaenoic acid | | | | |
| Prostaglandin | Prosta- | | 60203-57-8 | Prosta-5,9,13-trien-1-oic acid, 15-hydroxy-11- |
| J₂ | glandin | | | oxo-,(5Z,13E,15S) |
| 15-deoxy- | Prosta- | | 89886-60-2 | 11-oxo-prosta-5Z,9,12E,14Z-tetren-1-oic acid |
| Δ^{12,14}- | glandin J₂ analog | | | |
| prostaglandin | | | | |
| J₂ | | | | |
| Δ¹²- | Prosta- | | 87893-54-7 | Prosta-5,9,12-trien-1-oic acid, 15-hydroxy-11- |
| prostaglandin | glandin J₂ analog | | | oxo-,(5Z, 12E, 15S) |
| J₂ | | | | |

Notes:
^{a}N/A indicates that a common name for the compound is not known.

TABLE 2
Dosage and activity information about selected PPAR gamma agonists.

| COMPOUND^{a} | CITE | INDICATION | DOSAGE LEVEL | PPARγ^{b} EC₅₀ |
|---|---|---|---|---|
| (−)DRF2725 | Lohray et al., J. Med. | | | |
| | Chem., 44(16):2675-2678 | | | |
| | (2001) | | | |
| CS-011, CI- | Current Drugs Headline | Oral antidiabetic agent for | | 160 nM |
| 1037 | News, at http://www.current- | Type II, or adult-onset | | |
| | drugs.com/NEWS/ADA60- | diabetes. | | |
| | R1.htm, Oct. 09, 2001 | | | |
| AD-5075 | U.S. Pat. No. 5,972,881 | Non-insulin dependent | | |
| | | diabetes mellitis | | |
| BRL49653 | U.S. Pat. No. 6.191,154 | Alzheimer's disease, | | |
| | | central nervous system | | |
| | | injury | | |
| | U.S. Pat. No. 5,972,881 | Non-insulin dependent | 0.4-1 mg/kg · day | |
| | | diabetes mellitis | | |
| | Lehmann, J. M. et al., JBC | | | 0.03 μM and 0.1 μM |
| | Online, 270(22): 12953- | | | |
| | 12956 (1995) | | | |
| GW1929 | | | | |
| AY-31637 | U.S. Pat. No. 6,087,384 | apoptosis inhibitor | | |
| MCC-555 | U.S. Pat. No. 6,087,384 | apoptosis inhibitor | | |
| JTT501 | | | | |
| PD72953 | | | | |
| L-764406 | | | | |
| WAY-120,744 | | | | |
| G1262570X, | | | | |
| GG570 | | | | |
| Indomethacin | Lehmann, J. M. et al., JBC | | | 40 μM |
| | Online, 272(6):3406-3410 | | | |
| | (1997) | | | |
| Ciglitazone | T. M. Wilson et al., J. Med. | | | 3 μM |
| | Chem., 39:665 (1996) | | | |
| | U.S. Pat. No. 5,972,944 | Treatment of anovulation, | 0.01-20 | |
| | | hyperandrogenism, and | mg/kg · day | |
| | | hirsutism | | |
| | U.S. Pat. No. 6.191,154 | Alzheimer's disease, | | |
| | | central nervous system | | |
| | | injury | | |
| Darglitazone | U.S. Pat. No. 5,972,944 | Treatment of anovulation, | 0.01-20 | |
| | | hyperandrogenism, and | mg/kg · day | |
| | | hirsutism | | |
| | U.S. Pat. No. 6.191,154 | Alzheimer's disease, | | |
| | | central nervous system | | |
| | | injury | | |
| Englitazone | U.S. Pat. No. 6.191,154 | Alzheimer's disease, | | |
| | | central nervous system | | |
| | | injury | | |
| Pioglitazone | U.S. Pat. No. 5,972,944 | Treatment of anovulation, | 0.01-20 | |
| (Actos ®) | | hyperandrogenism, and | mg/kg · day | |
| | | hirsutism | | |
| | ACTOS ® (pioglitazone | Type 2 diabetes mellitis | 15-45 mg/day | |
| | hydrochloride) Complete | | | |
| | Prescribing Information, Eli | | | |
| | Lilly and Co., Indianapolis, | | | |
| | IN, November 1999. | | | |
| | U.S. Pat. No. 6.191,154 | Alzheimer's disease, | | |
| | | central nervous system | | |
| | | injury | | |
| | Lehmann, J. M. et al., JBC | | | 0.4 μM |
| | Online, 270(22): 12953- | | | |
| | 12956 (1995) | | | |
| Rosiglitazone | U.S. Pat. No. 5,972,944 | Treatment of anovulation, | 0.01-20 | 490 nM |
| (Avandia ®) | | hyperandrogenism, and | mg/kg · day | |
| | | hirsutism | | |
| | Prescribing Information, | Type 2 diabetes | 4-8 mg/day | |
| | AVANDIA ®, (rosiglitazone | | | |
| | maleate), SmithKline | | | |
| | Beecham Pharmaceuticals, | | | |
| | Philadelphia, PA, February 2001 | | | |
| | T. Fujiwara et al., Life Sci., | Type II diabetes | | |
| | 67:2405 (2000) | | | |
| Troglitazone | Y. Tsubouchi et al., | Inhibits growth of human | | |
| (Rezulin ®) | Biochem. biophys. Res. | cancer cells | | |
| | Communic., 270:400 | | | |
| | (2000); Y. F. Guan et al., | | | |
| | Neoplasia, 1:330 (1999); H. | | | |
| | Asou et al., Int. J. Oncol., | | | |
| | 15:1027 (1999) | | | |
| | T. Fujiwara et al., Life Sci., | Antiinflammatory and | | |
| | 67:2405 (2000) | antitumor activity | | |
| | U.S. Pat. No. 5,972,944 | Treatment of anovulation, | 0.01-20 | |
| | | hyperandrogenism, and | mg/kg · day | |
| | | hirsutism | | |
| | U.S. Pat. No. 6.191,154 | Alzheimer's disease, | | |
| | | central nervous system | | |
| | | injury | | |
| | U.S. Pat. No. 5,814,647 | The climacteric | 0.1-100 mg/day | |
| | | symptoms, cancer, | | |
| | | excessive uterine | | |
| | | bleeding | | |
| 5-[[4-[2- | U.S. Pat. No. 5,972,881 | | | |
| (methyl-2- | | | | |
| pyridinylamino) | | | | |
| ethoxy]phenyl] | | | | |
| methyl]-2,4- | | | | |
| thiazolidinedione | | | | |
| Docosahexaenoic | Bundy, G.L. et al., J. Med. | Platelet aggregation | | |
| kacid | Chem., 26:790-799 (1983) | inhibition | | |
| Prostaglandin | Fukushima, M., | Anti-tumor and anti-viral | | |
| J₂ | Eicosanoids, 3:189-199 | activity | | |
| | (1990). | | | |
| 15-deoxy- | Kliewer, S. A. et al., Cell, | Adipocyte differentiation | | |
| Δ^{12,14}- | 83:813-819 (1995). | promoter | | |
| prostaglandin | | | | |
| J₂ | | | | |
| Δ¹²- | Fukushima, M., | Anti-tumor and anti-viral | | |
| prostaglandin | Eicosanoids, 3:189-199 | activity | | |
| J₂ | (1990). | | | |
| One or more | U.S. Pat. No. 5,925,657 | Inflammatory bowel | at least 200 | |
| PPARγ | | disease, | mg/day; | |
| Agonists in | | immunodeficiency | Preferably, at | |
| general | | syndrome, multiple | least 800 mg/day | |
| | | sclerosis, cachexia | | |
| | U.S. Pat. Nos. 5,972,881 and | Non-insulin dependent | | |
| | 6,228,862 | diabetes | | |
| | U.S. Pat. No. 6,242,196 | Inhibiting tumor cell | | |
| | | growth | | |
| | U.S. Pat. No. 5,489,602 | Hypoglycemia and | | |
| | | hypolipidemia | | |
| | Neve, B. P. et al., | Atherosclerosis | | |
| | Biochemical Pharmacology, | | | |
| | 60:1245-1250 (2000). | | | |
| | Kliewer, et al., Recent Prog. | atherosclerosis, | | |
| | Horm. Res., 56:239-263 | dyslipidemia, obesity, | | |
| | (2001) | type-2 diabetes | | |

Notes:
^{a}Experimental compound number is provided if a common name for the compound is not known.
^{b}When two EC₅₀ values are given, the first value is for the PPARγ1 isoform and the second is for the PPARγ2 isoform.

Compounds that can act as the PPARγ agonist of the present invention are described in U.S. Pat. No. 6,200,998, which describes arylthiazolidinedione derivatives that can act as agonists for PPARα, PPARβ and PPARγ. The compounds are described as having the structure of formula X:

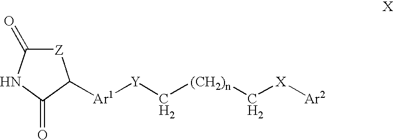

wherein

Ar¹ is (1) arylene or

(2) heteroarylene,

wherein arylene and heteroarylene are optionally substituted with from 1 to 4 groups selected from R^{a};

Ar² is (1) ortho-substituted aryl or

(2) ortho-substituted heteroaryl,

wherein said ortho substituent is selected from R; and aryl and heteroaryl are optionally further substituted with from 1-4 groups independently selected from R^{a};

X and Y are independently O, S, N—R^{b}, or CH₂;

Z is O or S;

n is 0 to 3;

R is (1) C₃₋₁₀ alkyl optionally substituted with 1-4 groups selected from halo and C₃₋₆ cycloalkyl,

(2) C₃₋₁₀ alkenyl, or

(3) C₃₋₈ cycloalkyl;

R^{a} is (1) C₁₋₅ alkanoyl,

(2) C₁₋₅ alkyl,

(3) C₂₋₁₅ alkenyl,

(4) C₂₋₁₅ alkynyl,

(5) halo,

(6) OR^{b},

(7) aryl, or

(8) heteroaryl,

wherein said alkyl, alkenyl, alkynyl, and alkanoyl are optionally substituted with from 1-5 groups selected from R^{c}, and said aryl and heteroaryl optionally substituted with 1 to 5 groups selected from R^{d};

R^{b} is (1) hydrogen,

(2) C₁₋₁₀ alkyl,

(3) C₂₋₁₀ alkenyl,

(4) C₂₋₁₀ alkynyl,

(5) aryl,

(6) heteroaryl,

(7) aryl C₁₋₅ alkyl,

(8) heteroaryl C₁₋₅ alkyl,

(9) C₁₋₅ cycloalkyl,

(10) C₃₋₈ cycloalkyl,

wherein alkyl, alkenyl, alkynyl are optionally substituted with one to four substituents independently selected from R^{c}, and cycloalkyl, aryl, and heteroaryl are optionally substituted with one to four substituents independently selected from R^{d}; or

R^{c} is (1) halo,

(2) aryl,

(3) heteroaryl,

(4) CN,

(5) NO₂,

(6) OR^{f},

(7) S(O)ₘR^{f}, m=0, 1 or 2, provided that R^{f} is not H when m is 1 or 2;

(8) NR^{f}R^{f},

(9) NR^{f}COR^{f},

(10) NR^{f}CO₂R^{f},

(11) NR^{f}CON(R^{f})₂,

(12) NR^{f}SO₂R^{f}, provided that R^{f} is not H,

(13) COR^{f},

(14) CO₂R^{f},

(15) CON(R^{f})₂,

(16) SO₂N(R^{f})₂,

(17) OCON(R^{f})₂, or

(18) C₃₋₈ cycloalkyl,

wherein said cycloalkyl, aryl and heteroaryl are optionally substituted with 1 to 3 groups of halo or C₁₋₆ alkyl;

R^{d} is (1) a group selected from R^{c},

(2) C₁₋₁₀ alkyl,

(3) C₂₋₁₀ alkenyl,

(3) C₂₋₁₀ alkenyl,

(4) C₂₋₁₀ alkynyl,

(5) aryl C₁₋₁₀ alkyl, or

(6) heteroaryl C₁₋₁₀ alkyl,

wherein alkyl, alkenyl, alkynyl, aryl, heteroaryl are optionally substituted with a group independently selected from R^{e};

R^{e} is (1) halogen,

(2) amino,

(3) carboxyl,

(4) C₁₋₄ alkyl,

(5) C₁₋₄ alkoxy,

(6) hydroxy,

(7) aryl,

(8) aryl C₁₋₄ alkyl, or

(9) aryloxy;

R^{f} is (1) hydrogen,

(2) C₁₋₁₀ alkyl,

(3) C₂₋₁₀ alkenyl,

(4) C₂₋₁₀ alkynyl,

(5) aryl,

(6) heteroaryl,

(7) aryl C₁₋₁₅ alkyl,

(8) heteroaryl C₁₋₁₅ alkyl,

(9) C₁₋₁₅ alkanoyl,

(10) C₃₋₈ cycloalkyl;

wherein alkyl, alkenyl, alkynyl, aryl, heteroaryl, alkanoyl and cycloalkyl are optionally substituted with one to four groups selected from R^{e};

or a pharmaceutically acceptable salt thereof.

PPARγ agonists that are useful in the present invention can be supplied by any source as long as the PPARγ agonist is pharmaceutically acceptable. PPARγ agonists can be isolated and purified from natural sources or can be synthesized. PPARγ agonists are preferably of a quality and purity that is conventional in the trade for use in pharmaceutical products.

Another component of the combination of the present invention is a cycloxygenase-2 selective inhibitor. The terms “cyclooxygenase-2 selective inhibitor”, or “Cox-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase . activity. Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or a pharmaceutically acceptable salt or prodrug thereof.

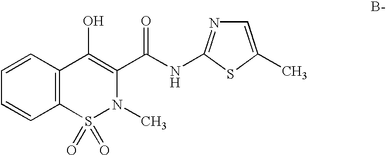

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

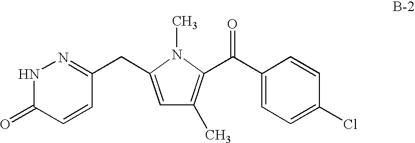

In a another embodiment of the invention the cyclooxygenase-2 selective inhibitor is of the chromene/chroman structural class that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, III, IV, V, and VI, shown below, and possessing, by way of example and not limitation, the structures disclosed in Table 3, including the diastereomers, enantiomers, racemates, tautomers, salts, esters, amides and prodrugs thereof.

Benzopyrans that can serve as a cyclooxygenase-2 selective inhibitor of the present invention include substituted benzopyran derivatives that are described in U.S. Pat. No. 6,271,253. One such class of compounds is defined by the general formula shown below in formulas I:

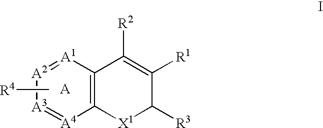

wherein X¹ is selected from O, S, CR^{c}R^{b} and NR^{a};

wherein R^{a} is selected from hydrido, C₁-C₃-alkyl, (optionally substituted phenyl)-C₁-C₃-alkyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;

wherein R¹ is selected from carboxyl, aminocarbonyl, C₁-C₆-alkylsulfonylaminocarbonyl and C₁-C₆-alkoxycarbonyl; wherein R² is selected from hydrido, phenyl, thienyl, C₁-C₆-alkyl and C₂-C₆-alkenyl;

wherein R³ is selected from C₁-C₃-perfluoroalkyl, chloro, C₁-C₆-alkylthio, C₁-C₆-alkoxy, nitro, cyano and cyano-C₁-C₃-alkyl;

wherein R⁴ is one or more radicals independently selected from hydrido, halo, C₁-C₆-alkyl, C₂-C₆-alkenyl, C₂-C₆-alkynyl, halo-C₂ -C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C6-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and

wherein the A ring atoms A¹, A², A³ and A⁴ are independently selected from carbon and nitrogen with the proviso that at least two of A¹, A², A³ and A⁴ are carbon;

or wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

Another class of benzopyran derivatives that can serve as the Cox-2 selective inhibitor of the present invention includes a compound having the structure of formula II:

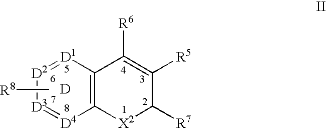

wherein X² is selected from O, S, CR^{c}R^{b} and NR^{a};

wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{c}R^{b} form a cyclopropyl ring;

wherein R⁵ is selected from carboxyl, aminocarbonyl, C₁-C₆-alkylsulfonylaminocarbonyl and C₁-C₆-alkoxycarbonyl;

wherein R⁶ is selected from hydrido, phenyl, thienyl, C₂-C₆-alkynyl and C₂-C₆-alkenyl;

wherein R⁷ is selected from C₁-C₃ -perfluoroalkyl, chloro, C₁-C₆-alkylthio, C₁-C₆-alkoxy, nitro, cyano and cyano-C₁-C₃-alkyl; wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂ O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆- haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the D ring atoms D¹, D², D³ and D⁴ are independently selected from carbon and nitrogen with the proviso that at least two of D¹, D², D³ and D⁴ are carbon; or

wherein R⁸ together with ring D forms a radical selected from naphthyl, quinolyl, isoquinolyl, quinolizinyl, quinoxalinyl and dibenzofuryl; or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

Formula III is:

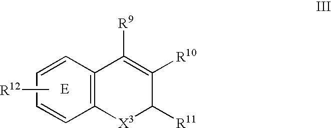

wherein X³ is selected from the group consisting of O or S or NR^{a};

wherein R^{a} is alkyl;

wherein R⁹ is selected from the group consisting of H and aryl;

wherein R¹⁰ is selected from the group consisting of carboxyl, aminocarbonyl, alkylsulfonylaminocarbonyl and alkoxycarbonyl; wherein R¹¹ is selected from the group consisting of haloalkyl, alkyl, aralkyl, cycloalkyl and aryl optionally substituted with one or more radicals selected from alkylthio, nitro and alkylsulfonyl; and

wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or

wherein R¹² together with ring E forms a naphthyl radical; or an isomer or pharmaceutically acceptable salt thereof; and including the diastereomers, enantiomers, racemates, tautomers, salts, esters, amides and prodrugs thereof.

A related class of compounds useful as cyclooxygenase-2 selective inhibitors in the present invention is described by Formulas IV and V:

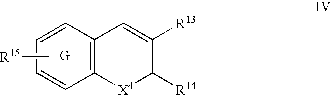

wherein X⁴ is selected from O or S or NR^{a};

wherein R^{a} is alkyl;

wherein R¹³ is selected from carboxyl, aminocarbonyl, alkylsulfonylaminocarbonyl and alkoxycarbonyl;

wherein R¹⁴ is selected from haloalkyl, alkyl, aralkyl, cycloalkyl and aryl optionally substituted with one or more radicals selected from alkylthio, nitro and alkylsulfonyl; and

wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or wherein R¹⁵ together with ring G forms a naphthyl radical; or an isomer or pharmaceutically acceptable salt thereof.

Formula V is:

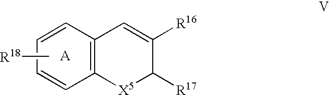

wherein:

X⁵ is selected from the group consisting of O or S or NR^{b};

R^{b} is alkyl;

R¹⁶ is selected from the group consisting of carboxyl, aminocarbonyl, alkylsulfonylaminocarbonyl and alkoxycarbonyl;

R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of lower haloalkyl, lower cycloalkyl and phenyl; and

R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R¹⁸ together with ring A forms a naphthyl radical; or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is carboxyl;

R¹⁷ is lower haloalkyl; and

R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or

wherein R² together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting trifluoromethyl and pentafluoroethyl; and

R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;

or an isomer or prodrug thereof.

The cyclooxygenase-2 selective inhibitor of the present invention can also be a compound having the structure of Formula VI:

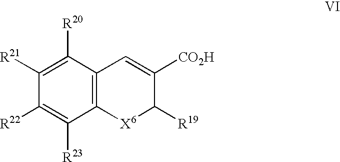

wherein:

X⁶ is selected from the group consisting of O and S;

R¹⁹ is lower haloalkyl;

R²⁰ is selected from the group consisting of hydrido, and halo;

R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R²² is selected from the group consisting of hydrido, lower alkyl, halo, lower alkoxy, and aryl; and

R²³ is selected from the group consisting of the group consisting of hydrido, halo, lower alkyl, lower alkoxy, and aryl;

or an isomer or prodrug thereof.

The cyclooxygenase-2 selective inhibitor can also be a compound of having the structure of Formula VI, wherein:

X⁶ is selected from the group consisting of O and S;

R¹⁹ is selected from the group consisting of trifluoromethyl and pentafluoroethyl;

R²⁰ is selected from the group consisting of hydrido, chloro, and fluoro,

R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R²² is selected from the group consisting of hydrido, methyl, ethyl, isopropyl, tert-butyl, chloro, methoxy, diethylamino, and phenyl; and

R²³ is selected from the group consisting of hydrido, chloro, bromo, fluoro, methyl, ethyl, tert-butyl, methoxy, and phenyl;

or an isomer or prodrug thereof.

TABLE 3
Examples of Chromene Cox-2 Selective Inhibitors

| Compound | |
|---|---|
| Number | Structural Formula |
| B-3 | 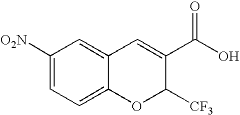 |
| | 6-Nitro-2-trifluoromethyl-2H-1- |
| | benzopyran-3-carboxylic acid |
| B-4 | 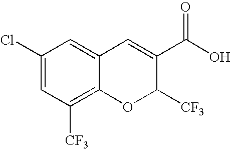 |
| | 6-Chloro-8-methyl-2-trifluoromethyl- |
| | 2H-1-benzopyran-3-carboxylic acid |
| B-5 | 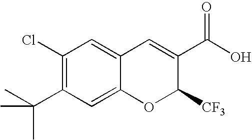 |
| | ((S)-6-Chloro-7-(1,1-dimethylethyl)-2-(trifluo |
| | romethyl-2H-1-benzopyran-3-carboxylic acid |
| B-6 | 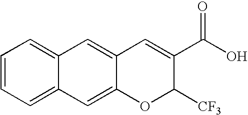 |
| | 2-Trifluoromethyl-2H-naphtho[2,3-b] |
| | pyran-3-carboxylic acid |
| B-7 | 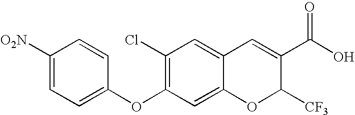 |
| | 6-Chloro-7-(4-nitrophenoxy)-2-(trifluoromethyl)-2H-1- |
| | benzopyran-3-carboxylic acid |
| B-8 | 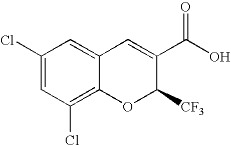 |
| | ((S)-6,8-Dichloro-2-(trifluoromethyl)- |
| | 2H-1-benzopyran-3-carboxylic acid |
| B-9 | 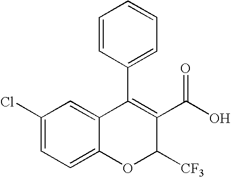 |
| | 6-Chloro-2-(trifluoromethyl)-4-phenyl-2H- |
| | 1-benzopyran-3-carboxylic acid |
| B-10 | 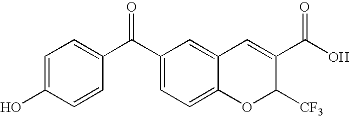 |
| | 6-(4-Hydroxybenzoyl)-2-(trifluoromethyl)- |
| | 2H-1-benzopyran-3-carboxylic acid |
| B-11 | 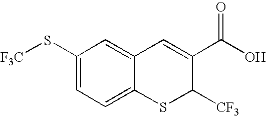 |
| | 2-(Trifluoromethyl)-6-[(trifluoromethyl)thio]- |
| | 2H-1-benzothiopyran-3-carboxylic acid |
| B-12 | 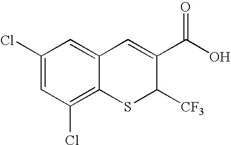 |
| | 6,8-Dichloro-2-trifluoromethyl-2H-1- |
| | benzothiopyran-3-Carboxylic acid |
| B-13 | 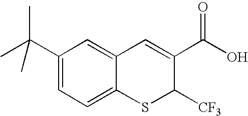 |
| | 6-(1,1-Dimethylethyl)-2-(trifluoromethyl)- |
| | 2H-1-benzothiopyran-3-carboxylic acid |
| B-14 | 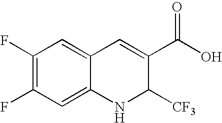 |
| | 6,7-Difluoro-1,2-dihydro-2-(trifluoro |
| | methyl)-3-quinolinecarboxylic acid |
| B-15 | 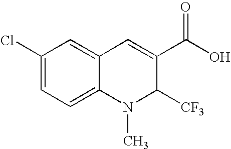 |
| | 6-Chloro-1,2-dihydro-1-methyl-2-(trifluoro |
| | methyl)-3-quinolinecarboxylic acid |
| B-16 | 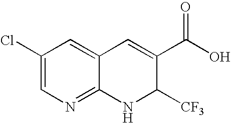 |
| | 6-Chloro-2-(trifluoromethyl)-1,2-dihydro |
| | [1,8]naphthyridine-3-carboxylic acid |
| B-17 | 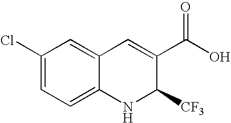 |
| | ((S)-6-Chloro-1,2-dihydro-2-(trifluoro |
| | methyl)-3-quinolinecarboxylic acid |

Examples of specific compounds that are useful for the cyclooxygenase-2 selective inhibitor include (without limitation):

a1) 8-acetyl-3-(4-fluorophenyl)-2-(4-methylsulfonyl)phenyl-imidazo( 1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-(1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4- (trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h10) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-difluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl) phenyl]oxazol-2-yl]-2-benzyl-acetate;

l8) 2-[4-(4-fluorophenyl)-5-[4-(methylsulfonyl)phenyl]oxazol-2-yl]acetic acid;

l9) 2-(tert-butyl)-4-(4-fluorophenyl)-5-[4-(methylsulfonyl)phenyl]oxazole;

l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2H-1-benzopyran-3-carboxylic acid;

m7) 6-chloro-8-(1-methylethyl)-2-trifluoromethyl-2H-1-benzopyran-3-carboxylic acid;

m8) 2-trifluoromethyl-3H-naphthopyran-3-carboxylic acid;

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the cyclooxygenase inhibitor can be selected from the class of tricyclic cyclooxygenase-2 selective inhibitors represented by the general structure of formula VII:

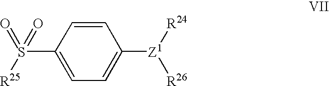

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula VII is selected from the group of compounds, illustrated in Table 4, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 4
Examples of Tricyclic COX-2 Selective Inhibitors

| Compound | |
|---|---|
| Number | Structural Formula |
| B-18 | 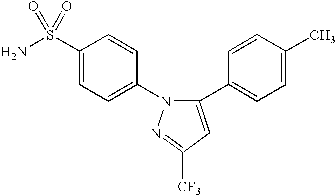 |
| B-19 | 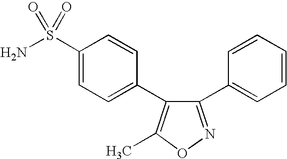 |
| B-20 | 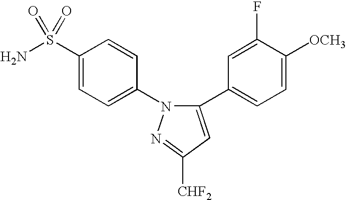 |
| B-21 | 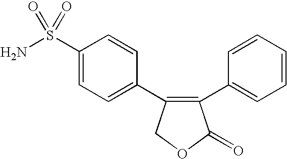 |
| B-22 | 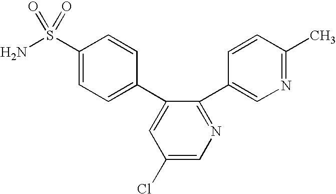 |
| B-23 | 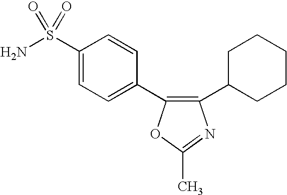 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic cyclooxygenase-2 selective inhibitor valdecoxib, B-19, (See, e.g., U.S. Pat. No. 5,633,272), may be advantageously employed as a source of a cyclooxygenase inhibitor.

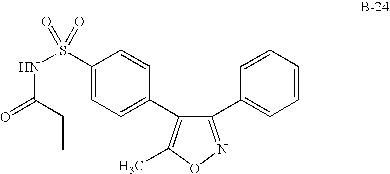

A preferred form of parecoxib is sodium parecoxib.

In another embodiment of the invention, the compound ABT-963 having the formula B-25 that has been previously described in International Publication number WO 00/24719, is another tricyclic cyclooxygenase-2 selective inhibitor which may be advantageously employed.

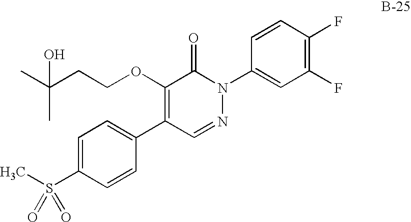

In a further embodiment of the invention, the cyclooxygenase inhibitor can be selected from the class of phenylacetic acid derivative cyclooxygenase-2 selective inhibitors represented by the general structure of Formula VIII:

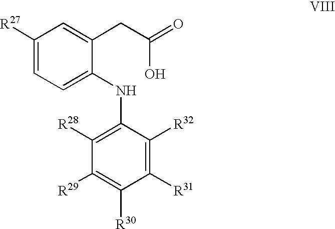

wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxy;

R³¹ is hydrogen, fluoro, or methyl; and

R³² is chloro, fluoro, trifluoromethyl, methyl, or ethyl, provided that R²⁸, R²⁹, R³⁰ and R³¹ are not all fluoro when R²⁷ is ethyl and R³⁰ is H.

A phenylacetic acid derivative cyclooxygenase-2 selective inhibitor that is described in WO 99/11605 is a compound that has the structure shown in Formula VIII,

wherein:

R²⁷ is ethyl;

R²⁸ and R³⁰ are chloro;

R²⁹ and R³¹ are hydrogen; and

R³² is methyl.

Another phenylacetic acid derivative cyclooxygenase-2 selective inhibitor is a compound that has the structure shown in Formula VIII,

wherein:

R²⁷ is propyl;

R²⁸ and R³⁰ are chloro;

R²⁹ and R³¹ are methyl; and

R³² is ethyl.

Another phenylacetic acid derivative cyclooxygenase-2 selective inhibitor that is described in WO 02/20090 is a compound that is referred to as COX-189 (also termed lumiracoxib), having CAS Reg. No. 220991-20-8, and having the structure shown in Formula VIII,

wherein:

R²⁷ is methyl;

R²⁸ is fluoro;

R³² is chloro; and

R²⁹, R³⁰, and R³¹ are hydrogen.

Compounds that have a structure similar to that shown in Formula VIII, which can serve as the Cox-2 selective inhibitor of the present invention, are described in U.S. Pat. Nos. 6,310,099, 6,291,523, and 5,958,978.

Other cyclooxygenase-2 selective inhibitors that can be used in the present invention have the general structure shown in formula IX, where the J group is a carbocycle or a heterocycle. Preferred embodiments have the structure:

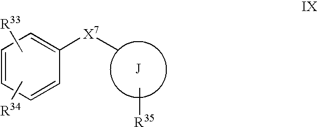

wherein:

X is O; J is 1-phenyl; R³³ is 2-NHSO₂CH₃; R³⁴ is 4-NO₂; and there is no R³⁵ group, (nimesulide), and

X is O; J is 1-oxo-inden-5-yl; R³³ is 2-F; R³⁴ is 4-F; and R³⁵ is 6-NHSO₂CH₃, (flosulide); and

X is O; J is cyclohexyl; R³³ is 2-NHSO₂CH₃; R³⁴ is 5-NO₂; and there is no R³⁵ group, (NS-398); and

X is S; J is 1-oxo-inden-5-yl; R³³ is 2-F; R³⁴ is 4-F; and R³⁵ is 6-N⁻ SO₂CH₃.Na⁺, (L-745337); and

X is S; J is thiophen-2-yl; R³³ is 4-F; there is no R³⁴ group; and R³⁵ is 5-NHSO₂CH₃, (RWJ-63556); and

X is O; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

Further information on the applications of the Cox-2 selective inhibitor N-(2-cyclohexyloxynitrophenyl) methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J. -P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

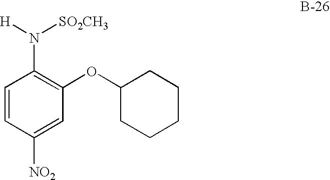

An evaluation of the anti-inflammatory activity of the cyclooxygenase-2 selective inhibitor, RWJ 63556, in a canine model of inflammation, was described by Kirchner et al., in J Pharmacol Exp Ther 282, 1094-1101 (1997).

Materials that can serve as the cyclooxygenase-2 selective inhibitor of the present invention include diarylmethylidenefuran derivatives that are described in U.S. Pat. No. 6,180,651. Such diarylmethylidenefuran derivatives have the general formula shown below in formula X:

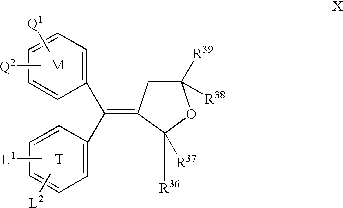

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms,

at least one of the substituents Q¹, Q², L¹ or L² is: an —S(O)ₙ—R group, in which n is an integer equal to 0, 1 or 2 and R is:

a lower alkyl radical having 1 to 6 carbon atoms or

a lower haloalkyl radical having 1 to 6 carbon atoms, or an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q¹ and Q² or L¹ and L² are a methylenedioxy group; and

R³⁶, R³⁷, R³⁸ and R³⁹ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R³⁶, R³⁷ or R³⁸, R³⁹ are an oxygen atom, or R³⁶, R³⁷ or R³⁸, R³⁹, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam1.htm, Oct. 4, 2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀ =0.39 mg/kg.

Compounds that may act as cyclooxygenase-2 selective inhibitors include multibinding compounds containing from 2 to 10 ligands covanlently attached to one or more linkers, as described in U.S. Pat. No. 6,395,724.

Compounds that may act as cyclooxygenase-2 inhibitors include conjugated linoleic acid that is described in U.S. Pat. No. 6,077,868.

Materials that can serve as a cyclooxygenase-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI:

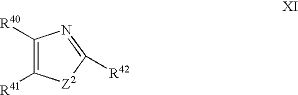

wherein:

Z² is an oxygen atom;

one of R⁴⁰ and R⁴¹ is a group of the formula

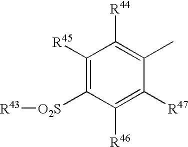

wherein:

R⁴³ is lower alkyl, amino or lower alkylamino; and

R⁴⁴, R⁴⁵, R⁴⁶ and R⁴⁷ are the same or different and each is hydrogen atom, halogen atom, lower alkyl, lower alkoxy, trifluoromethyl, hydroxy or amino, provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and

R³⁰ is a lower alkyl or a halogenated lower alkyl, and a pharmaceutically acceptable salt thereof.

Cox-2 selective inhibitors that are useful in the subject method and compositions can include compounds that are described in U.S. Pat. Nos. 6,080,876 and 6,133,292, and described by formula XII:

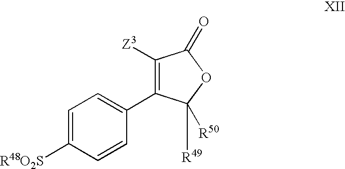

wherein:

Z³ is selected from the group consisting of:

(a) linear or branched C₁₋₆ alkyl,

(b) linear or branched C₁₋₆ alkoxy,

(c) unsubstituted, mono-, di- or tri-substituted phenyl or naphthyl wherein the substituents are selected from the group consisting of:

(1) hydrogen,

(2) halo,

(3) C₁₋₃ alkoxy,

(4) CN,

(5) C₁₋₃ fluoroalkyl

(6) C₁₋₃ alkyl,

(7) —CO₂H;

R⁴⁸ is selected from the group consisting of NH₂ and CH₃,

R⁴⁹ is selected from the group consisting of:

C₁₆ alkyl unsubstituted or substituted with C₃₋₆ cycloalkyl, and C₃₋₆ cycloalkyl;

R⁵⁰ is selected from the group consisting of:

C₁₋₆ alkyl unsubstituted or substituted with one, two or three fluoro atoms; and

C₃₋₆ cycloalkyl;

with the proviso that R⁴⁹ and R⁵⁰ are not the same.

Materials that can serve as cyclooxygenase-2 selective inhibitors include pyridines that are described in U.S. Pat. Nos. 6,369,275, 6,127,545, 6,130,334, 6,204,387, 6,071,936, 6,001,843 and 6,040,450, and which have the general formula described by formula XIII:

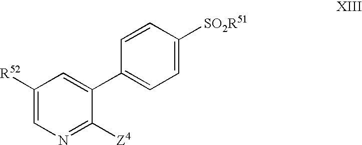

wherein:

R⁵¹ is selected from the group consisting of:

(a) CH₃,

(b) NH₂,

(c) NHC(O)CF₃,

(d) NHCH₃;

Z⁴ is a mono-, di-, or trisubstituted phenyl or pyridinyl (or the N-oxide thereof),

wherein the substituents are chosen from the group consisting of:

(a) hydrogen,

(b) halo,

(c) C₁₋₆ alkoxy,

(d) C₁₋₆ alkylthio,

(e) CN,

(f) C₁₋₆ alkyl,

(g) C₁₋₆ fluoroalkyl,

(h) N₃,

(i) —CO₂R⁵³,

(j) hydroxy,

(k) —C(R⁵⁴)(R⁵⁵)—OH,

(l) —C₁₋₆alkyl-CO₂—R⁵⁶,

(m) C₁₋₆fluoroalkoxy;

R⁵² is chosen from the group consisting of:

(a) halo,

(b) C₁₋₆alkoxy,

(c) C₁₋₆ alkylthio,

(d) CN,

(e) C₁₋₆ alkyl,

(f) C₁₋₆ fluoroalkyl,

(g) N₃,

(h) —CO₂R⁵⁷,

(i) hydroxy,

(j) —C(R⁵⁸)(R⁵⁹)—OH,

(k) —C₁₋₆alkyl-CO₂—R⁶⁰,

(l) C₁₋₆fluoroalkoxy,

(m) NO₂,

(n) NR⁶¹R⁶², and

(o) NHCOR⁶³;

R⁵³, R⁵⁴, R⁵⁵, R⁵⁶, R⁵⁷, R⁵⁸, R⁵⁹, R⁶⁰, R⁶¹, R⁶², R⁶³, are each independently chosen from the group consisting of:

(a) hydrogen, and

(b) C₁₋₆alkyl;

or R⁵⁴ and R⁵⁵, R⁵⁸ and R⁵⁹ or R⁶¹ and R⁶² together with the atom to which they are attached form a saturated monocyclic ring of 3, 4, 5, 6, or 7 atoms.

Materials that can serve as the cyclooxygenase-2 selective inhibitor of the present invention include diarylbenzopyran derivatives that are described in U.S. Pat. No. 6,340,694. Such diarylbenzopyran derivatives have the general formula shown below in formula XIV:

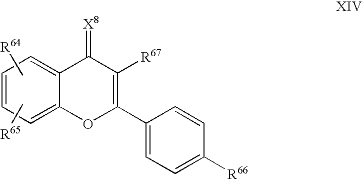

wherein:

X⁸ is an oxygen atom or a sulfur atom;

R⁶⁴ and R⁶⁵, identical to or different from each other, are independently a hydrogen atom, a halogen atom, a C₁-C₆ lower alkyl group, a trifluoromethyl group, an alkoxy group, a hydroxy group, a nitro group, a nitrile group, or a carboxyl group;

R⁶⁶ is a group of a formula: S(O)ₙR⁶⁸ wherein n is an integer of 0˜2, R⁶⁸ is a hydrogen atom, a C₁-C₆ lower alkyl group, or a group of a formula: NR⁶⁹ R⁷⁰ wherein R⁶⁹ and R⁷⁰, identical to or different from each other, are independently a hydrogen atom, or a C₁-C₆ lower alkyl group; and

R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures:

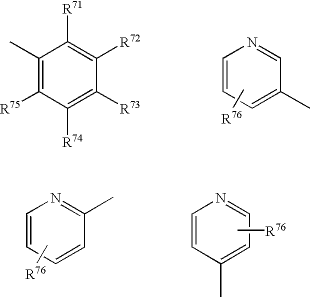

wherein:

R⁷¹ through R⁷⁵, identical to or different from one another, are independently a hydrogen atom, a halogen atom, a C₁-C₆ lower alkyl group, a trifluoromethyl group, an alkoxy group, a hydroxy group, a hydroxyalkyl group, a nitro group, a group of a formula: S(O)NR⁶⁸, a group of a formula: NR⁶⁹ R⁷⁰, a trifluoromethoxy group, a nitrile group a carboxyl group, an acetyl group, or a formyl group,

wherein n, R⁶⁸, R⁶⁹ and R⁷⁰ have the same meaning as defined by R⁶⁶ above; and

R⁷⁶ is a hydrogen atom, a halogen atom, a C₁-C₆ lower alkyl group, a trifluoromethyl group, an alkoxy group, a hydroxy group, a trifluoromethoxy group, a carboxyl group, or an acetyl group.

Materials that can serve as the cyclooxygenase-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV:

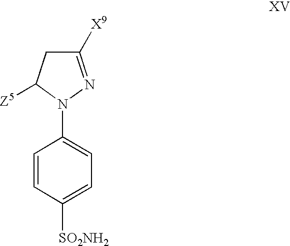

wherein:

X⁹ is selected from the group consisting of C₁-C₆ trihalomethyl, preferably trifluoromethyl; C₁-C₆ alkyl; and an optionally substituted or di-substituted phenyl group of formula XVI:

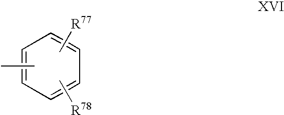

wherein:

R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;

Z⁵ is selected from the group consisting of substituted and unsubstituted aryl.

Materials that can serve as the cyclooxygenase-2 selective inhibitor of the present invention include heterocycles that are described in U.S. Pat. No. 6,153,787. Such heterocycles have the general formulas shown below in formulas XVII and XVIII:

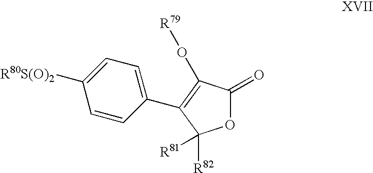

wherein:

R⁷⁹ is a mono-, di-, or tri-substituted C₁₋₁₂ alkyl, or a mono-, or an unsubstituted or mono-, di- or tri-substituted linear or branched C₂₋₁₀ alkenyl, or an unsubstituted or mono-, di- or tri-substituted linear or branched C₂₋₁₀ alkynyl, or an unsubstituted or mono-, di- or tri-substituted C₃₋₁₂ cycloalkenyl, or an unsubstituted or mono-, di- or tri-substituted C₅₋₁₂ cycloalkynyl, wherein the substituents are chosen from the group consisting of:

(a) halo, selected from F, Cl, Br, and I,

(b) OH,

(c) CF₃,

(d) C₃₋₆ cycloalkyl,

(e) ═O,

(f) dioxolane,

(g) CN; and

R⁸⁰ is selected from the group consisting of:

(a) CH₃,

(b) NH₂,

(c) NHC(O)CF₃,

(d) NHCH₃;

R⁸¹ and R⁸² are independently chosen from the group consisting of:

(a) hydrogen,

(b) C₁₋₁₀ alkyl;

or R⁸¹ and R⁸² together with the carbon to which they are attached form a saturated monocyclic carbon ring of 3, 4, 5, 6 or 7 atoms.

Formula XVIII is:

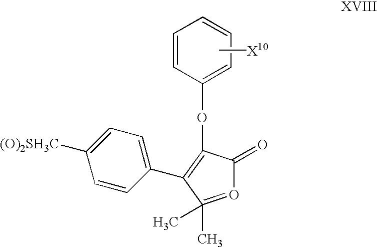

X¹⁰ is fluoro or chloro.

Materials that can serve as the cyclooxygenase-2 selective inhibitor of the present invention include 2,3,5-trisubstituted pyridines that are described in U.S. Pat. No. 6,046,217. Such pyridines have the general formula shown below in formula XIX:

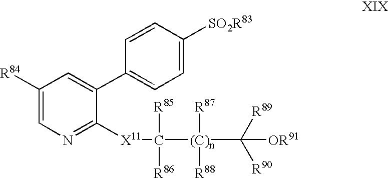

or a pharmaceutically acceptable salt thereof,

wherein:

X¹¹ is selected from the group consisting of:

(a) O,

(b) S,

(c) bond;

n is 0 or 1;

R⁸³ is selected from the group consisting of:

(a) CH₃,

(b) NH₂,

(c) NHC(O)CF₃;

R⁸⁴ is chosen from the group consisting of:

(a) halo,

(b) C₁₋₆ alkoxy,

(c) C₁₋₆ alkylthio,

(d) CN,

(e) C₁₋₆ alkyl,

(f) C₁₋₆ fluoroalkyl,

(g) N₃,

(h) —CO₂ R⁹²,

(i) hydroxy,

(j) —C(R⁹³)(R⁹⁴)—OH,

(k) —C₁₋₆ alkyl-CO₂—R⁹⁵,

(i) C₁₋₆ fluoroalkoxy,

(m) NO₂,

(n) NR⁹⁶ R⁹⁷,

(o) NHCOR⁹⁸;

R⁸⁵ to R⁹⁸ are independantly chosen from the group consisting of

(a) hydrogen,

(b) C₁₋₆ alkyl;

or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

One preferred embodiment of the Cox-2 selective inhibitor of formula XIX is that wherein X is a bond.

Another preferred embodiment of the Cox-2 selective inhibitor of formula XIX is that wherein X is O.

Another preferred embodiment of the Cox-2 selective inhibitor of formula XIX is that wherein X is S.

Another preferred embodiment of the Cox-2 selective inhibitor of formula XIX is that wherein R⁸³ is CH₃.

Another preferred embodiment of the Cox-2 selective inhibitor of formula XIX is that wherein R⁸⁴ is halo or C₁₋₆ fluoroalkyl.

Materials that can serve as the cyclooxygenase-2 selective inhibitor of the present invention include diaryl bicyclic heterocycles that are described in U.S. Pat. No. 6,329,421. Such diaryl bicyclic heterocycles have the general formula shown below in formula XX:

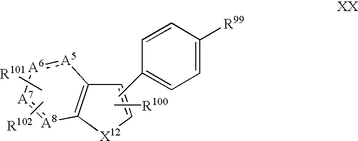

and pharmaceutically acceptable salts thereof wherein:

—A⁵═A⁶—A⁷═A⁸— is selected from the group consisting of:

(a) —CH═CH—CH═CH—,

(b) —CH₂—CH₂—CH₂—C(O)—, —CH₂—CH₂—C(O)—CH₂—, —CH₂—C(O)—CH₂—CH₂, —C(O)—CH₂—CH₂—CH₂,

(c) —CH₂—CH₂—C(O)—, —CH₂—C(O)—CH₂—, —C(O)—CH₂ —CH₂—

(d) —CH₂—CH₂—O—C(O)—, CH₂—O—C(O)—CH₂—, —O—C(O)—CH₂—CH₂—,

(e) —CH₂—CH₂—C(O)—O—, —CH₂—C(O)—OCH₂—C(O)—O—CH₂—CH₂—,

(f) —C(R¹⁰⁵)₂—O—C(O)—, —C(O)—O—C(R¹⁰⁵)₂—, —O—C(O)—C(R¹⁰⁵)₂—, —C(R¹⁰⁵)₂—C(O)—O—,

(g) —N═CH—CH═CH—,

(h) —CH═N—CH═CH—,

(i) —CH═CH—N═CH—,

(j) —CH═CH—CH═N—,

(k) —N═CH—CH═N—,

(l) —N═CH—N═CH—,

(m) —CH═N—CH═N—,

(n) —S—CH═N—,

(o) —S—N═CH—,

(p) —N═N—NH—,

(q) —CH═N—S—, and

(r) —N═CH—S—;

R⁹⁹ is selected from the group consisting of:

(a) S(O)₂ CH₃,

(b) S(O)₂ NH₂,

(c) S(O)₂ NHCOCF₃,

(d) S(O)(NH)CH₃,

(e) S(O)(NH)NH₂,

(f) S(O)(NH)NHCOCF₃,

(g) P(O)(CH₃)OH, and

(h) P(O)(CH₃)NH₂;

R¹⁰⁰ is selected from the group consisting of:

(a) C₁₋₆ alkyl,

(b) C₃₋₇, cycloalkyl,

(c) mono- or di-substituted phenyl or naphthyl wherein the substituent is selected from the group consisting of:

(1) hydrogen,

(2) halo, including F, Cl, Br, I,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹⁰³)(R¹⁰⁴)—OH,

(12) —C(R¹⁰³)(R¹⁰⁴)—O—C₁₋₄ alkyl, and

(13) —C₁₋₆ alkyl-CO₂—R¹⁰⁶;

(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹⁰³)(R¹⁰⁴)—OH, and

(10) —C(R¹⁰³)(R¹⁰⁴)—O—C₁₋₄ alkyl;

(e) benzoheteroaryl which includes the benzo fused analogs of (d); R¹⁰¹ and R¹⁰² are the substituents residing on any position of —A⁵═A⁶—A⁷=A⁸— and are selected independently from the group consisting of:

(a) hydrogen,

(b) CF₃,

(c) CN,

(d) C₁₋₆ alkyl,

(e) —Q³ wherein Q³ is Q⁴, CO₂H, C(R¹⁰³)(R¹⁰⁴)OH,

(f) —O-Q⁴,

(g) —S-Q⁴, and

(h) optionally substituted:

(1) —C₁₋₅ alkyl-Q³,

(2) —O—C₁₋₅ alkyl-Q³,

(3) —S—C₁₋₅ alkyl-Q³,

(4) —C₁₋₃ alkyl-O—C₁₋₃ alkyl-Q³,

(5) —C₁₋₃ alkyl-S—C₁₋₃ alkyl-Q³,

(6) —C₁₋₅ alkyl-O-Q⁴,

(7) —C₁₋₅ alkyl-S-Q⁴,

wherein the substituent resides on the alkyl chain and the substituent is C₁₋₃ alkyl, and Q³ is Q⁴, CO₂H, C(R¹⁰³)(R¹⁰⁴)OH Q⁴ is CO₂ —C₁₋₄ alkyl, tetrazolyl-5-yl, or C(R¹⁰³)(R¹⁰⁴)O—C₁₋₄ alkyl;

R¹⁰³, R¹⁰⁴ and R¹⁰⁵ are each independently selected from the group consisting of

(a) hydrogen,

(b) C₁₋₆ alkyl; or

R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;

R¹⁰⁶ is hydrogen or C₁₋₆ alkyl;

R¹⁰⁷ is hydrogen, C₁₋₆ alkyl or aryl;

X⁷ is O, S, NR¹⁰⁷, CO, C(R¹⁰⁷)₂, C(R¹⁰⁷)(OH), —C(R¹⁰⁷)═C(R¹⁰⁷)—; —C(R¹⁰⁷)═N—; —N═C(R¹⁰⁷)—.

Compounds that may act as cyclooxygenase-2 inhibitors include salts of 5-amino or a substituted amino 1,2,3-triazole compound that are described in U.S. Pat. No. 6,239,137. The salts are of a class of compounds of formula XXI:

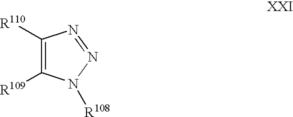

wherein:

R¹⁰⁸ is:

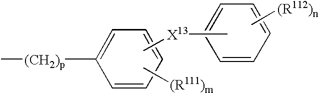

wherein:

p is 0 to 2; m is 0 to 4; and n is 0 to 5; X¹³ is O, S, SO, SO₂, CO, CHCN, CH₂ or C═NR¹¹³ where R¹¹³ is hydrogen, loweralkyl, hydroxy, loweralkoxy, amino, loweralkylamino, diloweralkylamino or cyano; and, R¹¹¹ and R¹¹² are independently halogen, cyano, trifluoromethyl, loweralkanoyl, nitro, loweralkyl, loweralkoxy, carboxy, lowercarbalkoxy, trifuloromethoxy, acetamido, loweralkylthio, loweralkylsulfinyl, loweralkylsulfonyl, trichlorovinyl, trifluoromethylthio, trifluoromethylsulfinyl, or trifluoromethylsulfonyl; R¹⁰⁹ is amino, mono or diloweralkyl amino, acetamido, acetimido, ureido, formamido, formamido or guanidino; and R¹¹⁰ is carbamoyl, cyano, carbazoyl, amidino or N-hydroxycarbamoyl; wherein the loweralkyl, loweralkyl containing, loweralkoxy and loweralkanoyl groups contain from 1 to 3 carbon atoms.

Materials that can serve as a cyclooxygenase-2 selective inhibitor of the present invention include pyrazole derivatives that are described in U.S. Pat. No. 6,136,831. Such pyrazole derivatives have the formula shown below in formula XXII:

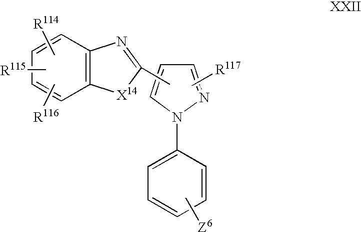

wherein:

R¹¹⁴ is hydrogen or halogen, R¹¹⁵ and R¹¹⁶ are each independently hydrogen, halogen, lower alkyl, lower alkoxy, hydroxy or lower alkanoyloxy;

R¹¹⁷ is lower haloalkyl or lower alkyl;

X¹⁴ is sulfur, oxygen or NH; and

Z⁶ is lower alkylthio, lower alkylsulfonyl or sulfamoyl; or a pharmaceutically acceptable salt thereof.

Materials that can serve as a cyclooxygenase-2 selective inhibitor of the present invention include substituted derivatives of benzosulphonamides that are described in U.S. Pat. No. 6,297,282. Such benzosulphonamide derivatives have the formula shown below in formula XXIII:

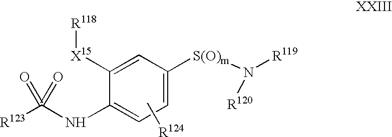

wherein:

X¹⁵ denotes oxygen, sulphur or NH;

R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;

R¹¹⁹ and R¹²⁰, independently from one another, denote hydrogen, an optionally polyfluorised alkyl group, an aralkyl, aryl or heteroaryl group or a group (CH₂)ₙ—X¹⁶; or

R¹¹⁹ and R¹²⁰, together with the N— atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶; X¹⁶ denotes halogen, NO₂, —OR¹²¹, —COR¹²¹, —CO₂R¹²¹, —OCO₂R¹²¹, —CN, —CONR¹²¹OR¹²², —CONR¹²¹R¹²², —SR¹²¹, —S(O)R¹²¹, —S(O)₂ R¹²¹, —NR¹²¹R¹²², —NHC(O)R¹²¹, —NHS(O)₂R¹²¹; n denotes a whole number from 0 to 6;

R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;

R¹²⁴ denotes halogen, hydroxy, a straight-chained or branched alkyl, alkoxy, acyloxy or alkyloxycarbonyl group with 1-6 C-atoms, which can optionally be mono- or polysubstituted by halogen, NO₂, —OR¹²¹, —COR¹²¹, —CO₂R¹²¹, —OCO₂R¹²¹, —CN, —CONR¹²¹, OR¹²², —CONR¹²¹ R¹²², —SR¹²¹, —S(O)R¹²¹, —S(O)₂R¹²¹, —NR¹²¹R¹²², —NHC(O)R¹²¹, —NHS(O)₂R¹²¹, or a polyfluoroalkyl group;

R¹²¹ and R¹²², independently from one another, denote hydrogen, alkyl, aralkyl or aryl; and

m denotes a whole number from 0 to 2;

and the pharmaceutically-acceptable salts thereof.

Materials that can serve as a cyclooxygenase-2 selective inhibitor of the present invention include 3-phenyl-4-(4(methylsulfonyl)phenyl)-2-(5H)-furanones that are described in U.S. Pat. No. 6,239,173. Such 3-phenyl-4-(4(methylsulfonyl)phenyl)-2-(5H)-furanones have the formula shown below in formula XXIV:

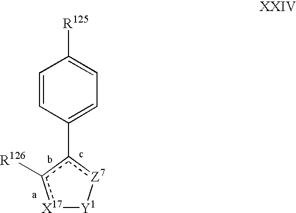

or pharmaceutically acceptable salts thereof wherein:

X¹⁷—Y¹-Z⁷-is selected from the group consisting of:

(a) —CH₂ CH₂ CH₂—,

(b) —C(O)CH₂ CH₂—,

(c) —CH₂ CH₂ C(O)—,

(d) —CR¹²⁹ (R^{129′})—O—C(O)—,

(e) —C(O)—O—CR¹²⁹(R^{129′})—,

(f) —CH₂—NR¹²⁷—CH₂—,

(g) —CR¹²⁹ (R^{129′})—NR¹²⁷—C(O)—,

(h) —CR¹²⁸═CR^{128′}—S—,

(i) —S—CR¹²⁸═CR^{128′}—,

(j) —S—N═CH—,

(k) —CH═N—S—,

(l) —N═CR¹²⁸—O—,

(m) —O—CR4═N—,

(n) —N═CR¹²⁸—NH—,

(o) —N═CR¹²⁸—S—, and

(p) —S—CR¹²⁸═N—,

(q) —C(O)—NR¹²⁷—CR¹²⁹(R^{129′})—,

(r) —R¹²⁷N—CH═CH— provided R₁₂₂ is not —S(O)₂CH₃,

(s) —CH═CH—NR¹²⁷— provided R¹²⁵ is not —S(O)₂CH₃, when side b is a double bond, and sides a and c are single bonds; and

X¹⁷—Y¹-Z⁷-is selected from the group consisting of:

(a) ═CH—O—CH═, and

(b) ═CH—NR¹²⁷—CH═,

(c) ═N—S—CH═,

(d) ═CH—S—N═,

(e) ═N—O—CH═,

(f) ═CH—O—N═,

(g) ═N—S—N═,

(h) ═N—O—N═, when sides a and c are double bonds and side b is a single bond;

R¹²⁵ is selected from the group consisting of:

(a) S(O)₂CH₃,

(b) S(O)₂NH₂,

(c) S(O)₂NHC(O)CF₃,

(d) S(O)(NH)CH₃,

(e) S(O)(NH)NH₂,

(f) S(O)(NH)NHC(O)CF₃,

(g) P(O)(CH₃)OH, and

(h) P(O)(CH₃)NH₂;

R¹²⁶ is selected from the group consisting of

(a) C₁₋₆ alkyl,

(b) C₃, C₄, C₅, C₆, and C₇, cycloalkyl,

(c) mono-, di- or tri-substituted phenyl or naphthyl, wherein the substituent is selected from the group consisting of:

(1) hydrogen,

(2) halo,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹²⁹)(R¹³⁰)—OH,

(12) —C(R¹²⁹)(R¹³⁰)—O—C₁₋₄ alkyl, and

(13) —C₁₋₆ alkyl-CO₂—R¹²⁹;

(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms, or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹²⁹)(R¹³⁰)—OH, and

(10) —C(R¹²⁹)(R¹³⁰)—O—C₁₋₄ alkyl;

(e) benzoheteroaryl which includes the benzo fused analogs of (d);

R¹²⁷ is selected from the group consisting of:

(a) hydrogen,

(b) CF₃,

(c) CN,

(d) C₁₋₆ alkyl,

(e) hydroxyC₁₋₆ alkyl,

(f) —C(O)—C₁₋₆ alkyl,

(g) optionally substituted:

(1) —C₁₋₅ alkyl-Q⁵,

(2) —C₁₋₃ alkyl-O—C₁₋₃ alkyl-Q⁵,

(3) —C₁₋₃ alkyl-S—C₁₋₃ alkyl-Q⁵,

(4) —C₁₋₅ alkyl-O-Q⁵, or

(5) —C₁₋₅ alkyl-S-Q⁵,

wherein the substituent resides on the alkyl and the substituent is C₁₋₃ alkyl;

(h) —Q⁵;

R^{128 ′} and R¹²⁸ are each independently selected from the group consisting of:

(a) hydrogen,

(b) CF₃,

(c) CN,

(d) C₁₋₆ alkyl,

(e) —Q⁵,

(f) —O-Q⁵;

(g) —S-Q⁵, and

(h) optionally substituted:

(1) —C₁₋₅ alkyl-Q⁵,

(2) —O—C₁₋₅ alkyl-Q⁵,

(3) —S—C₁₋₅ alkyl-Q⁵,

(4) —C₁₋₃ alkyl-O—C₁₋₃ alkyl-Q⁵,

(5) —C₁₋₃ alkyl-S—C₁₋₃ alkyl-Q⁵,

(6) —C₁₋₅ alkyl-O-Q⁵,

(7) —C₁₋₅ alkyl-S-Q⁵,

wherein the substituent resides on the alkyl and the substituent is C₁₋₃ alkyl, and

R¹²⁹, R^{129′}, R¹³⁰, R¹³¹ and R¹³² are each independently selected from the group consisting of:

(a) hydrogen,

(b) C₁₋₆ alkyl;

or R¹²⁹ and R¹³⁰ or R¹³¹ and R¹³² together with the carbon to which they are attached form a saturated monocyclic carbon ring of 3, 4, 5, 6 or 7 atoms;

Q⁵ is CO₂H, CO₂—C₁₋₄ alkyl, tetrazolyl-5-yl, C(R¹³¹)(R¹³²)(OH), or C(R¹³¹)(R¹³²)(O—C₁₋₄ alkyl);

provided that when X—Y—Z is —S—CR¹²⁸═CR^{128′}, then R¹²⁸ and R^{128′} are other than CF₃.

Materials that can serve as a cyclooxygenase-2 selective inhibitor of the present invention include bicycliccarbonyl indole compounds that are described in U.S. Pat. No. 6,303,628. Such bicycliccarbonyl indole compounds have the formula shown below in formula XXV:

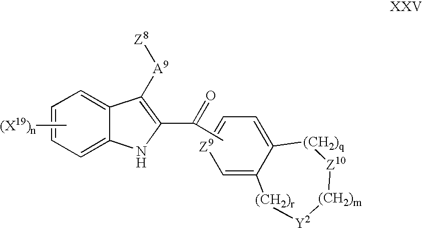

or the pharmaceutically acceptable salts thereof wherein

A⁹ is C₁₋₆ alkylene or —NR¹³³—;

Z⁸ is C(=L³)R¹³⁴, or SO₂R¹³⁵;

Z⁹ is CH or N;

Z¹⁰ and Y² are independently selected from —CH₂—, O, S and —N—R¹³³;

m is 1, 2 or 3;

q and r are independently 0, 1 or 2;

X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄ alkyl)amino and cyano;

n is 0, 1, 2, 3 or 4;

L³ is oxygen or sulfur;

R¹³³ is hydrogen or C₁₋₄ alkyl;

R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₄ alkyl(C₃₋₇ cycloalkoxy), —NR¹³⁶R¹³⁷, C₁₋₄ alkylphenyl-O— or phenyl-O—, said phenyl being optionally substituted with one to five substituents independently selected from halogen, C₁₋₄ alkyl, hydroxy, C₁₋₄ alkoxy and nitro;

R¹³⁵ is C₁₋₆ alkyl or halo-substituted C₁₋₆ alkyl; and

R¹³⁶ and R¹³⁷ are independently selected from hydrogen, C₁₋₆ alkyl and halo-substituted C₁₋₆ alkyl.

Materials that can serve as a cyclooxygenase-2 selective inhibitor of the present invention include benzimidazole compounds that are described in U.S. Pat. No. 6,310,079. Such benzimidazole compounds have the formula shown below in formula XXVI:

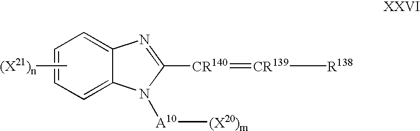

or a pharmaceutically acceptable salt thereof, wherein:

A¹⁰ is heteroaryl selected from a 5-membered monocyclic aromatic ring having one hetero atom selected from O, S and N and optionally containing one to three N atom(s) in addition to said hetero atom, or a 6-membered monocyclic aromatic ring having one N atom and optionally containing one to four N atom(s) in addition to said N atom; and said heteroaryl being connected to the nitrogen atom on the benzimidazole through a carbon atom on the heteroaryl ring;

X²⁰ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N, N-di(C₁-C₄ alkyl)amino, [N-(C₁-C₄ alkyl)amino]C₁-C₄ alkyl, [N, N-di(C₁-C₄ alkyl)amino]C₁-C₄ alkyl, N—(C₁-C₄ alkanoyl)amonio, N—(C₁-C₄ alkyl)(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N, N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N, N-di(C₁-C₄ alkyl)amino]sulfonyl; X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N, N-di(C₁-C₄ alkyl)amino, [N—(C₁-C₄ alkyl)amino]C₁-C₄ alkyl, [N, N-di(C₁-C₄ alkyl)amino]C₁-C₄ alkyl, N—(C₁-C₄ alkanoyl)amino, N-(C₁-C₄ alkyl)-N—(C₁-C₄ alkanoyl) amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)cabonyl, cabamoyl, [N—(C₁-C₄ alkyl) amino]carbonyl, [N, N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N, N-di(C₁-C₄ alkyl)amino]sulfonyl;

R¹³⁸ is selected from hydrogen,

straight or branched C₁-C₄ alkyl optionally substituted with one to three substituent(s) wherein said substituents are independently selected from halo hydroxy, C₁-C₄ alkoxy, amino, N—(C₁-C₄ alkyl)amino and N, N-di(C₁-C₄ alkyl)amino,

C₃-C₈ cycloalkyl optionally substituted with one to three substituent(s) wherein said substituents are indepently selected from halo, C₁-C₄ alkyl, hydroxy, C₁-C₄ alkoxy, amino, N—(C₁-C₄ alkyl)amino and N, N-di(C₁-C₄ alkyl)amino,

C₄-C₈ cycloalkenyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N, N-di(C₁-C₄ alkyl)amino,

phenyl optionally substituted with one to three substituent(s) wherein said substituents are independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N, N-di(C₁-C₄ alkyl)amino, [N—(C₁-C₄ alkyl)amino]C₁-C₄ alkyl, [N, N-di(C₁-C₄ alkyl)amino]C₁-C₄ alkyl, N—(C₁-C₄ alkanoyl)amino, N—[C₁-C₄ alkyl)(C₁-C₄ alkanoyl)]amino, N—[(C₁-C₄ alkyl)sulfony]amino, N-[(halo-substituted C₁-C₄ alkyl)sulfonyl]amino, C₁-C₄ alkanoyl, carboxy, (C₁-C₄ alkoxy)carbonyl, carbomoyl, [N—(C₁-C₄ alky)amino]carbonyl, [N, N di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N, N-di(C₁-C₄ alkyl)amino]sulfonyl; and

heteroaryl selected from:

a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and

said heteroaryl being optionally substituted with one to three substituent(s) selected from X²⁰;

R¹³⁹ and R¹⁴⁰ are independently selected from:

hydrogen,

halo,

C₁-C₄ alkyl,

phenyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N, N-di(C₁-C₄ alkyl)amino,

or R¹³⁸ and R¹³⁹ can form, together with the carbon atom to which they are attached, a C₃-C₇ cycloalkyl ring;

m is 0, 1, 2, 3, 4 or 5; and

n is 0, 1, 2, 3 or 4.

Materials that can serve as a cyclooxygenase-2 selective inhibitor of the present invention include indole compounds that are described in U.S. Pat. No. 6,300,363. Such indole compounds have the formula shown below in formula XXVII:

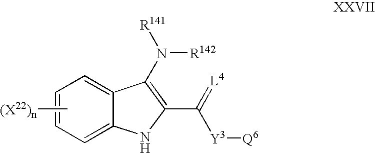

and the pharmaceutically acceptable salts thereof,

wherein:

L⁴ is oxygen or sulfur;

Y³ is a direct bond or C₁₋₄ alkylidene;

Q⁶ is:

(a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino,

(b) C₃₋₇ cycloalkyl optionally substituted with up to three substituents independently selected from hydroxy, C₁₋₄ alkyl and C₁₋₄ alkoxy,

(c) phenyl or naphthyl, said phenyl or naphthyl being optionally substituted with up to four substituents independently selected from: (c-1) halo, C₁₋₄ alkyl, halosubstituted C₁₋₄ alkyl, hydroxy, C₁₋₄ alkoxy, halosubstituted C₁₋₄ alkoxy, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂ H, CO₂(C₁₋₄ alkyl), C₁₋₄ alkyl-OH, C₁₋₄ alkyl-OR¹⁴³, CONH₂, CONH(C₁₋₄ alkyl), CON(C₁₋₄alkyl)₂ and —O—Y-phenyl, said phenyl being optionally substituted with one or two substituents independently selected from halo, C₁₋₄ alkyl, CF₃, hydroxy, OR¹⁴³, S(O)ₘR¹⁴³, amino, mono- or di-(C₁₋₄ alkyl)amino and CN;

(d) a monocyclic aromatic group of 5 atoms, said aromatic group having one heteroatom selected from O, S and N and optionally containing up to three N atoms in addition to said heteroatom, and said aromatic group being substituted with up to three substitutents independently selected from:

(d-1) halo, C₁₋₄ alkyl, halosubstituted C₁₋₄ alkyl, hydroxy, C₁₋₄ alkoxy, halosubstituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³;

(e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);

R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂H, CO₂(C₁₋₄ alkyl), CONH₂, CONH(C₁₋₄ alkyl) and CON(C₁₋₄ alkyl)₂;

R¹⁴² is:

(a) hydrogen,

(b) C₁₋₄ alkyl,

(c) C(O)R¹⁴⁵,

wherein R¹⁴⁵ is selected from:

(c-1) C₁₋₂₂ alkyl or C₂₋₂₂ alkenyl, said alkyl or alkenyl being optionally substituted with up to four substituents independently selected from: (c-1-1) halo, hydroxy, OR¹⁴³, S(O)ₘ R¹⁴³, nitro, amino, mono- or di-(C₁₋₄ alkyl)amino, NHSO₂R¹⁴³, CO₂H, CO₂ (C₁₋₄ alkyl), CONH₂, CONH(C₁₋₄ alkyl), CON(C₁₋₄ alkyl)₂, OC(O)R¹⁴³, thienyl, naphthyl and groups of the following formulae:

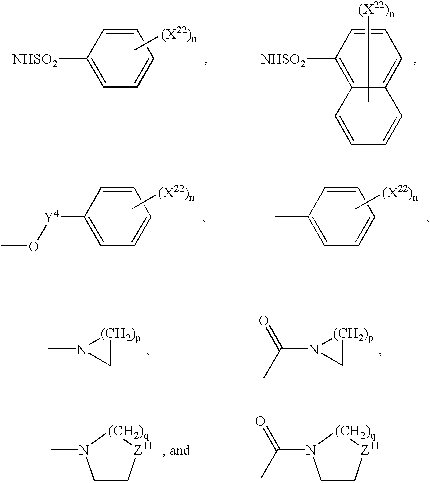

(c-2) C₁₋₂₂ alkyl or C₂₋₂₂ alkenyl, said alkyl or alkenyl being optionally substituted with five to forty-five halogen atoms,

(c-3) —Y⁵—C₃₋₇ cycloalkyl or —Y⁵—C₃₋₇ cycloalkenyl, said cycloalkyl or cycloalkenyl being optionally substituted with up to three substituent independently selected from:

(c-3-1) C₁₋₄ alkyl, hydroxy, OR¹⁴³, S(O)ₘR¹⁴³, amino, mono or di-(C₁₋₄ alkyl)amino, CONH₂, CONH(C₁₋₄ alkyl) and CON(C₁₋₄ alkyl)₂, (c-4) phenyl or naphthyl, said phenyl or naphthyl being optionally substituted with up to seven (preferably up to seven) substituents independently selected from:

(c-4-1) halo, C₁₋₈ alkyl, C₁₋₄ alkyl-OH, hydroxy, C₁₋₈ alkoxy, halosubstituted C₁₋₈ alkyl, halosubstituted C₁₋₈ alkoxy, CN, nitro, S(O)ₘR¹⁴³, SO₂ NH₂, SO₂NH(C₁₋₄ alkyl), SO₂N(C₁₋₄ alkyl)₂, amino, C₁₋₄ alkylamino, di-(C₁₋₄ alkyl)amino, CONH₂, CONH(C₁₋₄ alkyl), CON(C₁₋₄ alkyl)₂, OC(O)R¹⁴³, and phenyl optionally substituted with up to three substituents independently selected from halo, C₁₋₄ alkyl, hydroxy, OCH₃, CF₃, OCF₃, CN, nitro, amino, mono- or di-(C₁₋₄ alkyl)amino, CO₂H, CO₂(C₁₋₄ alkyl) and CONH₂,

(c-5) a monocyclic aromatic group as defined in (d) and (e) above, said aromatic group being optionally substituted with up to three substituents independently selected from:

(c-5-1) halo, C₁₋₈ alkyl, C₁₋₄ alkyl-OH, hydroxy, C₁₋₈ alkoxy, CF₃, OCF₃, CN, nitro, S(O)ₘR¹⁴³, amino, mono- or di-(C₁₋₄ alkyl)amino, CONH₂, CONH(C₁₋₄ alkyl), CON(C₁₋₄ alkyl)₂, CO₂H and CO₂(C₁₋₄ alkyl), and —Y-phenyl, said phenyl being optionally substituted with up to three substituents independently selected halogen, C₁₋₄ alkyl, hydroxy, C₁₋₄ alkoxy, CF₃, OCF₃, CN, nitro, S(O)ₘR¹⁴³, amino, mono- or di-(C₁₋₄ alkyl)amino, CO₂H, CO₂(C₁₋₄ alkyl), CONH₂, CONH(C₁₋₄ alkyl) and CON(C₁₋₄ alkyl)₂,

(c-6) a group of the following formula:

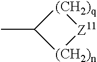

X²² is halo, C₁₋₄ alkyl, hydroxy, C₁₋₄ alkoxy, halosubstitutued C₁₋₄ alkoxy, S(O)ₘR¹⁴³, amino, mono- or di-(C₁₋₄ alkyl)amino, NHSO₂R¹⁴³, nitro, halosubstitutued C₁₋₄ alkyl, CN, CO₂H, CO₂ (C₁₋₄ alkyl), C₁₋₄ alkyl-OH, C₁₋₄ alkylOR¹⁴³, CONH₂, CONH(C₁₋₄ alkyl) or CON(C₁₋₄ alkyl)₂; R¹⁴³ is C₁₋₄ alkyl or halosubstituted C₁₋₄ alkyl; p1 m is 0, 1 or 2; n is 0, 1, 2 or 3; p is 1, 2, 3, 4 or 5; q is 2 or 3; Z¹¹ is oxygen, sulfur or NR¹⁴⁴; and

R¹⁴⁴ is hydrogen, C₁₋₆ alkyl, halosubstitutued C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘ R¹⁴³, amino, mono- or di-(C₁₋₄ alkyl)amino, CF₃, OCF₃, CN and nitro;

with the proviso that a group of formula —Y⁵-Q is not methyl or ethyl when X²² is hydrogen;

L⁴ is oxygen;

R¹⁴¹ is hydrogen; and

R¹⁴² is acetyl.

Materials that can serve as a cyclooxygenase-2 selective inhibitor of the present invention include aryl phenylhydrazides that are described in U.S. Pat. No. 6,077,869. Such aryl phenylhydrazides have the formula shown below in formula XXVIII:

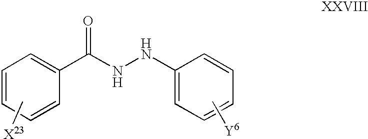

wherein:

X²³ and Y⁶ are selected from hydrogen, halogen, alkyl, nitro, amino or other oxygen and sulfur containing functional groups such as hydroxy, methoxy and methylsulfonyl.

Materials that can serve as a cyclooxygenase-2 selective inhibitor of the present invention include 2-aryloxy, 4-aryl furan-2-ones that are described in U.S. Pat. No. 6,140,515. Such 2-aryloxy, 4-aryl furan-2-ones have the formula shown below in formula XXIX:

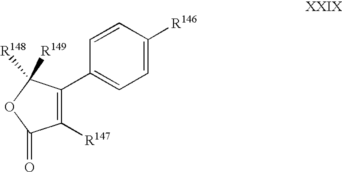

or a pharmaceutical salt thereof,

wherein:

R¹⁴⁶ is selected from the group consisting of SCH₃, —S(O)₂CH₃ and —S(O)₂NH₂;

R¹⁴⁷ is selected from the group consisting of OR¹⁵⁰, mono or di-substituted phenyl or pyridyl wherein the substituents are selected from the group consisting of methyl, chloro and F;

R¹⁵⁰ is unsubstituted or mono or di-substituted phenyl or pyridyl wherein the substituents are selected from the group consisting of methyl, chloro and F;

R¹⁴⁸ is H, C₁₋₄ alkyl optionally substituted with 1 to 3 groups of F, Cl or Br; and

R¹⁴⁹ is H, C₁₋₄ alkyl optionally substituted with 1 to 3 groups of F, Cl or Br, with the proviso that R¹⁴⁸ and R¹⁴⁹ are not the same.

Materials that can serve as a cyclooxygenase-2 selective inhibitor of the present invention include bisaryl compounds that are described in U.S. Pat. No. 5,994,379. Such bisaryl compounds have the formula shown below in formula XXX:

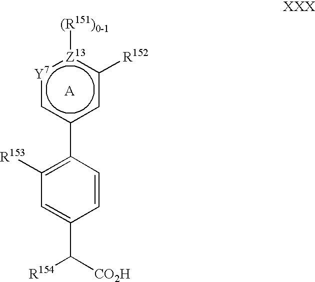

or a pharmaceutically acceptable salt, ester or tautomer thereof,

wherein:

Z¹³ is C or N;

when Z¹³ is N, R¹⁵¹ represents H or is absent, or is taken in conjunction with R¹⁵² as described below:

when Z¹³ is C, R¹⁵¹ represents H and R¹⁵² is a moiety which has the following characteristics:

(a) it is a linear chain of 3-4 atoms containing 0-2 double bonds, which can adopt an energetically stable transoid configuration and if a double bond is present, the bond is in the trans configuration,

(b) it is lipophilic except for the atom bonded directly to ring A, which is either lipophilic or non-lipophilic, and

(c) there exists an energetically stable configuration planar with ring A to within about 15 degrees;

or R¹⁵¹ and R¹⁵² are taken in combination and represent a 5- or 6-membered aromatic or non-aromatic ring D fused to ring A, said ring D containing 0-3 heteroatoms selected from O, S and N;

said ring D being lipophilic except for the atoms attached directly to ring A, which are lipophilic or non-lipophilic, and said ring D having available an energetically stable configuration planar with ring A to within about 15 degrees;

said ring D further being substituted with 1 R^{a} group selected from the group consisting of: C₁₋₂ alkyl, —OC₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;

Y⁷ represents N, CH or C—OC₁₋₃ alkyl, and when Z¹³ is N, Y⁷ can also represent a carbonyl group;

R¹⁵³ represents H, Br, Cl or F; and

R¹⁵⁴ represents H or CH₃.

Materials that can serve as a cyclooxygenase-2 selective inhibitor of the present invention include 1,5-diarylpyrazoles that are described in U.S. Pat. No. 6,028,202. Such 1,5-diarylpyrazoles have the formula shown below in formula XXXI:

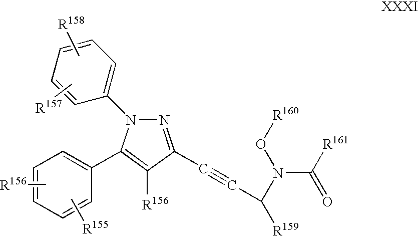

wherein:

R¹⁵⁵, R¹⁵⁶, R¹⁵⁷, and R¹⁵⁸ are independently selected from the groups consisting of hydrogen, C₁₋₅ alkyl, C₁₋₅ alkoxy, phenyl, halo, hydroxy, C₁₋₅ alkylsulfonyl, C₁₋₅ alkylthio, trihaloC₁₋₅ alkyl, amino, nitro and 2-quinolinylmethoxy;

R¹⁵⁹ is hydrogen, C₁₋₅ alkyl, trihaloC₁₋₅ alkyl, phenyl, substituted phenyl where the phenyl substitutents are halogen, C₁₋₅ alkoxy, trihaloC₁₋₅ alkyl or nitro or R¹⁵⁹ is heteroaryl of 5-7 ring members where at least one of the ring members is nitrogen, sulfur or oxygen;

R¹⁶⁰ is hydrogen, C₁₋₅ alkyl, phenyl C₁₋₅ alkyl, substituted phenyl C₁₋₅ alkyl where the phenyl substitutents are halogen, C₁₋₅ alkoxy, trihaloC₁₋₅ alkyl or nitro, or R¹⁶⁰ is C₁₋₅ alkoxycarbonyl, phenoxycarbonyl, substituted phenoxycarbonyl where the phenyl substitutents are halogen, C₁₋₅ alkoxy, trihaloC₁₋₅ alkyl or nitro;

R¹⁶¹ is C₁₋₁₀ alkyl, substituted C₁₋₁₀ alkyl where the substituents are halogen, trihaloC₁₋₅ alkyl, C₁₋₅ alkoxy, carboxy, C₁₋₅ alkoxycarbonyl, amino, C₁₋₅ alkylamino, diC₁₋₅ alkylamino, diC₁₋₅ alkylaminoC₁₋₅ alkylamino, C₁₋₅ alkylaminoC₁₋₅ alkylamino or a heterocycle containing 4-8 ring atoms where one more of the ring atoms is nitrogen, oxygen or sulfur, where said heterocycle may be optionally substituted with C₁₋₅ alkyl; or R¹⁶¹ is phenyl, substituted phenyl (where the phenyl substitutents are one or more of C₁₋₅ alkyl, halogen, C₁₋₅ alkoxy, trihaloC₁₋₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or

R¹⁶¹ is NR¹⁶³ R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁₋₅ alkyl;

R¹⁶² is hydrogen, C₁₋₅ alkyl, nitro, amino, and halogen;

and pharmaceutically acceptable salts thereof.

Materials that can serve as a cyclooxygenase-2 selective inhibitor of the present invention include 2-substituted imidazoles that are described in U.S. Pat. No. 6,040,320. Such 2-substituted imidazoles have the formula shown below in formula XXXII:

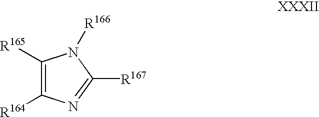

wherein:

R¹⁶⁴ is phenyl, heteroaryl wherein the heteroaryl contains 5 to 6 ring atoms, or

substituted phenyl;

wherein the substituents are independently selected from one or members of the group consisting of C₁₋₅ alkyl, halogen, nitro, trifluoromethyl and nitrile;

R¹⁶⁵ is phenyl, heteroaryl wherein the heteroaryl contains 5 to 6 ring atoms,

substituted heteroaryl;

wherein the substituents are independently selected from one or more members of the group consisting of C₁₋₅ alkyl and halogen, or substituted phenyl,

wherein the substituents are independently selected from one or members of the group consisting of C₁₋₅ alkyl, halogen, nitro, trifluoromethyl and nitrile;

R¹⁶⁶ is hydrogen, SEM, C₁₋₅ alkoxycarbonyl, aryloxycarbonyl, arylC₁₋₅alkyloxycarbonyl, arylC₁₋₅ alkyl, phthalimidoC₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms, or substituted arylC₁₋₅alkyl,

wherein the aryl substituents are independently selected from one or more members of the group consisting of C₁₋₅ alkyl, C₁₋₅ alkoxy, halogen, amino, C₁₋₅ alkylamino, and diC₁₋₅ alkylamino;

R¹⁶⁷ is (A¹¹)ₙ-(CH¹⁶⁵)_{q}—X²⁴ wherein:

A¹¹ is sulfur or carbonyl;

n is 0 or 1;

q is 0-9;

X²⁴ is selected from the group consisting of hydrogen, hydroxy, halogen, vinyl, ethynyl, C₁₋₅ alkyl, C₃₋₇ cycloalkyl, C₁₋₅ alkoxy, phenoxy, phenyl, arylC₁₋₅ alkyl, amino, C₁₋₅ alkylamino, nitrile, phthalimido, amido, phenylcarbonyl, C₁₋₅ alkylaminocarbonyl, phenylaminocarbonyl, arylC₁₋₅ alkylaminocarbonyl, C₁₋₅ alkylthio, C₁₋₅ alkylsulfonyl, phenylsulfonyl,

substituted sulfonamido,

wherein the sulfonyl substituent is selected from the group consisting of C₁₋₅ alkyl, phenyl, araC₁₋₅ alkyl, thienyl, furanyl, and naphthyl;

substituted vinyl,

wherein the substituents are independently selected from one or members of the group consisting of fluorine, bromine, chlorine and iodine,

substituted ethynyl,

wherein the substituents are independently selected from one or more members of the group consisting of fluorine, bromine chlorine and iodine,

substituted C₁₋₅ alkyl,

wherein the substituents are selected from the group consisting of one or more C₁₋₅ alkoxy, trihaloalkyl, phthalimido and amino, substituted phenyl,

wherein the phenyl substituents are independently selected from one or more members of the group consisting of C₁₋₅ alkyl, halogen and C₁₋₅ alkoxy,

substituted phenoxy,

wherein the phenyl substituents are independently selected from one or more members of the group consisting of C₁₋₅ alkyl, halogen and C₁₋₅ alkoxy,

substituted C₁₋₅ alkoxy,

wherein the alkyl substituent is selected from the group consisting of phthalimido and amino,

substituted arylC₁₋₅ alkyl,

wherein the alkyl substituent is hydroxyl,

substituted arylC₁₋₅ alkyl,

wherein the phenyl substituents are independently selected from one or more members of the group consisting of C₁₋₅ alkyl, halogen and C₁₋₅ alkoxy,

substituted amido,

wherein the carbonyl substituent is selected from the group consisting of C₁₋₅ alkyl, phenyl, arylC₁₋₅ alkyl, thienyl, furanyl, and naphthyl,

substituted phenylcarbonyl,

wherein the phenyl substituents are independently selected from one or members of the group consisting of C₁₋₅ alkyl, halogen and C₁₋₅ alkoxy,

substituted C₁₋₅ alkylthio,

wherein the alkyl substituent is selected from the group consisting of hydroxy and phthalimido,

substituted C₁₋₅ alkylsulfonyl,

wherein the alkyl substituent is selected from the group consisting of hydroxy and phthalimido,

substituted phenylsulfonyl,

wherein the phenyl substituents are independently selected from one or members of the group consisting of bromine, fluorine, chlorine, C₁₋₅ alkoxy and trifluoromethyl,

with the proviso:

if A¹¹ is sulfur and X²⁴ is other than hydrogen, C₁₋₅ alkylaminocarbonyl, phenylaminocarbonyl, arylC₁₋₅ alkylaminocarbonyl, C₁₋₅ alkylsulfonyl or phenylsulfonyl, then q must be equal to or greater than 1;

if A¹¹ is sulfur and q is 1, then X²⁴ cannot be C₁₋₂ alkyl;

if A¹¹ is carbonyl and q is 0, then X²⁴ cannot be vinyl, ethynyl, C₁₋₅ alkylaminocarbonyl, phenylaminocarbonyl, arylC₁₋₅ alkylaminocarbonyl,C₁₋₅ alkylsulfonyl or phenylsulfonyl;

if A¹¹ is carbonyl, q is 0 and X²⁴ is H, then R¹⁶⁶ is not SEM (2-(trimethylsilyl)ethoxymethyl);

if n is 0 and q is 0, then X²⁴ cannot be hydrogen;

and pharmaceutically acceptable salts thereof.

Materials that can serve as a cyclooxygenase-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV:

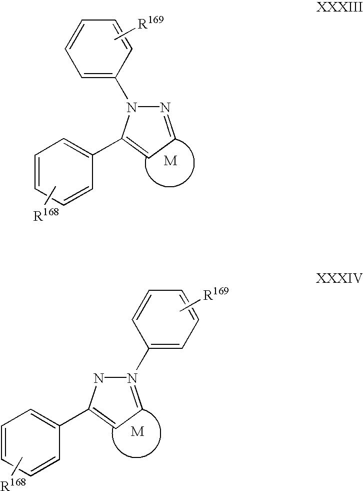

wherein:

R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂(C₁-C₆)alkyl; and the fused moiety M is a group selected from the group consisting of an optionally substituted cyclohexyl and cycloheptyl group having the formulae:

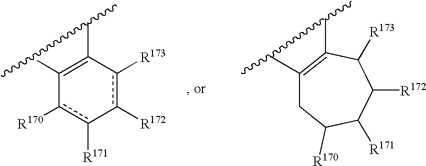

wherein:

R¹⁷⁰ is selected from the group consisting of hydrogen, halogen, hydroxy and carbonyl;

or R¹⁷⁰ and R¹⁷¹ taken together form a moiety selected from the group consisting of —OCOCH₂—, —ONH(CH₃)COCH₂—, —OCOCH.dbd. and —O—;

R¹⁷¹ and R¹⁷² are independently selected from the group consisting of hydrogen, halogen, hydroxy, carbonyl, amino, (C₁-C₆)alkyl, (C₁-C₆)alkoxy, ═NOH, —NR¹⁷⁴R¹⁷⁵, —OCH₃, —OCH₂CH₃, —OSO₂NHCO₂CH₃, ═CHCO₂CH₂CH₃, —CH₂CO₂H, —CH₂CO₂CH₃, —CH₂CO₂CH₂CH₃, —CH₂CON(CH₃)₂, —CH₂CO₂NHCH₃, —CHCHCO₂CH₂CH₃, —OCON(CH₃)OH, —C(COCH₃)₂, di(C₁-C₆)alkyl and di(C₁-C₆)alkoxy;

R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from the group consisting of halogen, hydroxy, amino, (C₁-C₆)alkyl and (C₁-C₆)alkoxy;

or R¹⁷² and R¹⁷³ taken together form a moiety selected from the group consisting of —O— and

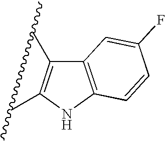

R¹⁷⁴ is selected from the group consisting of hydrogen, OH, —OCOCH₃, —COCH₃ and (C₁-C₆)alkyl; and

R¹⁷⁵ is selected from the group consisting of hydrogen, OH, —OCOCH₃, —COCH₃, (C₁-C₆)alkyl, —CONH₂ and —SO₂CH₃; with the proviso that

if M is a cyclohexyl group, then R¹⁷⁰ through R¹⁷³ may not all be hydrogen; and

pharmaceutically acceptable salts, esters and pro-drug forms thereof.

Materials that can serve as a cyclooxygenase-2 selective inhibitor of the present invention include esters derived from indolealkanols and novel amides derived from indolealkylamides that are described in U.S. Pat. No. 6,306,890. Such compounds have the general formula shown below in formula XXXV:

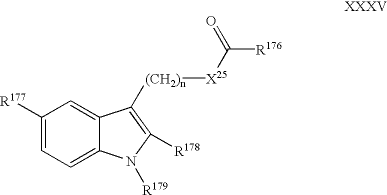

wherein:

R¹⁷⁶ is C₁ to C₆ alkyl, C₁ to C₆ branched alkyl, C₄ to C₈ cycloalkyl, C₁ to C₆ hydroxyalkyl, branched C₁ to C₆ hydroxyalkyl, hydroxy substituted C₄ to C₈ aryl, primary, secondary or tertiary C₁ to C₆ alkylamino, primary, secondary or tertiary branched C₁ to C₆ alkylamino, primary, secondary or tertiary C₄ to C₈ arylamino, C₁ to C₆ alkylcarboxylic acid, branched C₁ to C₆ alkylcarboxylic acid, C₁ to C₆ alkylester, branched C₁ to C₆ alkylester, C₄ to C₈ aryl, C₄ to C₈ arylcarboxylic acid, C₄ to C₈ arylester, C₄ to C₈ aryl substituted C₁ to C₆ alkyl, C₄ to C₈ heterocyclic alkyl or aryl with O, N or S in the ring, alkyl-substituted or aryl-substituted C₄ to C₈ heterocyclic alkyl or aryl with O, N or S in the ring, or halo-substituted versions thereof, where halo is chloro, bromo, fluoro or iodo;

R¹⁷⁷ is C₁ to C₆ alkyl, C₁ to C₆ branched alkyl, C₄ to C₈ cycloalkyl, C₄ to C₈ aryl, C₄ to C₈ aryl-substituted C₁ to C₆ alkyl, C₁ to C₆ alkoxy, C₁ to C₆ branched alkoxy, C₄ to C₈ aryloxy, or halo-substituted versions thereof or R¹⁷⁷ is halo where halo is chloro, fluoro, bromo, or iodo;

R¹⁷⁸ is hydrogen, C₁ to C₆ alkyl or C₁ to C₆ branched alkyl;

R¹⁷⁹ is C₁ to C₆ alkyl, C₄ to C₈ aroyl, C₄ to C₈ aryl, C₄ to C₈ heterocyclic alkyl or aryl with O, N or S in the ring, C₄ to C₈ aryl-substituted C₁ to C₆ alkyl, alkyl-substituted or aryl-substituted C₄ to C₈ heterocyclic alkyl or aryl with O, N or S in the ring, alkyl-substituted C₄ to C₈ aroyl, or alkyl-substituted C₄ to C₈ aryl, or halo-substituted versions thereof where halo is chloro, bromo, or iodo;

n is 1, 2, 3, or 4; and

X²⁵ is O, NH, or N—R¹⁸⁰, where R¹⁸⁰ is C₁ to C₆ alkyl or C₁ to C₆ branched alkyl.

Materials that can serve as a cyclooxygenase-2 selective inhibitor of the present invention include pyridazinone compounds that are described in U.S. Pat. No. 6,307,047. Such pyridazinone compounds have the formula shown below in formula XXXVI:

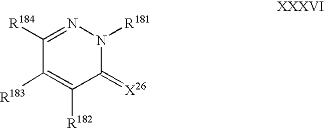

or a pharmaceutically acceptable salt, ester, or prodrug thereof,

wherein:

X²⁶ is selected from the group consisting of O, S, —NR¹⁸⁵, —NORₐ, and —NNR^{b}R^{c};

R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;

R^{a}, R^{b}, and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙC(O)R¹⁸⁶, —(CH₂)ₙCH(OH)R¹⁸⁶, —(CH₂)ₙC(NOR^{d})R¹⁸⁶, —(CH₂)ₙCH(NOR^{d})R¹⁸⁶, —(CH₂)ₙCH(NR^{d}R^{e})R¹⁸⁶, —R¹⁸⁷R¹⁸⁸, —(CH₂)ₙC□CR¹⁸⁸, —(CH₂)ₙ[CH(CX^{26′}₃)]ₘ(CH₂)ₚR¹⁸⁸, —(CH₂)ₙ(CX²⁶′₂)ₘ(CH₂)ₚR¹⁸⁸, and —(CH₂)ₙ(CHX²⁶′)ₘ(CH₂)ₘR¹⁸⁸;

R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;

R¹⁸⁷ is selected from the group consisting of alkenylene, alkylene, halo-substituted alkenylene, and halo-substituted alkylene;

R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;

R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;

X^{26′} is halogen;

m is an integer from 0-5;

n is an integer from 0-10; and

p is an integer from 0-10; and

R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴;

provided that one of R¹⁸², R¹⁸³, or R¹⁸⁴ must be Z¹⁴, and further provided that only one of R¹⁸², R¹⁸³, or R¹⁸⁴ is Z¹⁴;

Z¹⁴ is selected from the group consisting of:

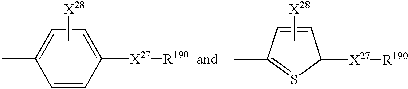

²⁷ is selected from the group consisting of S(O)₂, S(O)(NR¹⁹¹), S(O), Se(O)₂, P(O)(OR¹⁹²), and P(O)(NR¹⁹³R¹⁹⁴);

X²⁸ is selected from the group consisting of hydrogen, alkenyl, alkyl, alkynyl and halogen;

R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;

R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;

Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵, and —N(R¹⁹⁷)R¹⁹⁵;

R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹R²⁰⁰; and

R¹⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Materials that can serve as a cyclooxygenase-2 selective inhibitor of the present invention include benzosulphonamide derivatives that are described in U.S. Pat. No. 6,004,948. Such benzosulphonamide derivatives have the formula shown below in formula

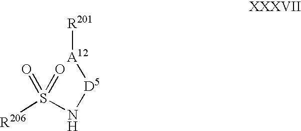

herein:

A¹² denotes oxygen, sulphur or NH;

R²⁰¹ denotes a cycloalkyl, aryl or heteroaryl group optionally mono- or polysubstituted by halogen, alkyl, CF₃ or alkoxy;

D⁵ denotes a group of formula XXXVIII or XXXIX:

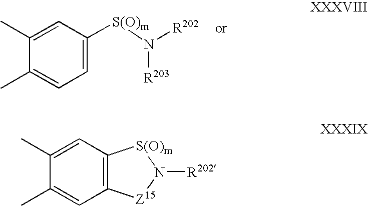

R²⁰² and R²⁰³ independently of each other denote hydrogen, an optionally polyfluorinated alkyl radical, an aralkyl, aryl or heteroaryl radical or a radical (CH₂)ₙ—X²⁹; or

R²⁰² and R²⁰³ together with the N-atom denote a three- to seven-membered, saturated, partially or totally unsaturated heterocycle with one or more heteroatoms N, O, or S, which may optionally be substituted by oxo, an alkyl, alkylaryl or aryl group or a group (CH₂)ₙ—X²⁹, R²⁰²′ denotes hydrogen, an optionally polyfluorinated alkyl group, an aralkyl, aryl or heteroaryl group or a group (CH₂)ₙ—X²⁹,

wherein:

X²⁹ denotes halogen, NO₂, —OR²⁰⁴, —COR²⁰⁴, —CO₂R²⁰⁴, —OCO₂R²⁰⁴, —CN, —CONR²⁰⁴OR²⁰⁵, —CONR²⁰⁴R²⁰⁵, —SR²⁰⁴, —S(O)R²⁰⁴, —S(O)₂R²⁰⁴, —NR²⁰⁴R²⁰⁵, —NHC(O)R²⁰⁴, —NHS(O)₂R²⁰⁴; Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂NH—, —N═CH—, —NHCH—, —CH₂—CH₂—NH—, —CH═CH—, >N—R²⁰³, >C═O, >S(O)ₘ;

R²⁰⁴ and R²⁰⁵ independently of each other denote hydrogen, alkyl, aralkyl or aryl;

n is an integer from 0 to 6;

R²⁰⁶ is a straight-chained or branched C₁₋₄-alkyl group which may optionally be mono- or polysubstituted by halogen or alkoxy, or R²⁰⁶ denotes CF₃; and

m denotes an integer from 0 to 2;

with the proviso that A¹² does not represent O if R²⁰⁶ denotes CF₃;

and the pharmaceutically acceptable salts thereof.

Cox-2 selective inhibitors that are useful in the subject method and compositions can include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

In an embodiment of the present method, a subject in need of prevention or treatment of pain, inflammation or inflammation-associated disorder is treated with a PPARγ agonist and a cyclooxygenase-2 selective inhibitor or prodrug thereof. In one embodiment, the subject is treated with an amount of a PPARγ agonist and an amount of a Cox-2 selective inhibitor, where the amount of the PPARγ agonist, when administered with the amount of the Cox-2 selective inhibitor, together provide a dosage or amount of the combination that is sufficient to constitute an effective amount of the combination. The effective amount can be a pain or inflammation suppressing treatment or prevention effective amount.

In another embodiment of the subject method, a subject in need of prevention or treatment of cardiovascular disease or disorder is treated with a PPARγ agonist and a cyclooxygenase-2 selective inhibitor or prodrug thereof. In one embodiment, the subject is treated with an amount of a PPARγ agonist and an amount of a Cox-2 selective inhibitor, where the amount of the PPARγ agonist, when administered with the amount of the Cox-2 selective inhibitor, together provide a dosage or amount of the combination that is sufficient to constitute an effective amount of the combination. The effective amount can be a cardiovascular disorder or disease suppressing treatment or prevention effective amount.

In another embodiment of the present method, a subject in need of prevention or treatment of cancer is treated with a PPARγ agonist and a cyclooxygenase-2 selective inhibitor or prodrug thereof. In one embodiment, the subject is treated with an amount of a PPARγ agonist and an amount of a Cox-2 selective inhibitor, where the amount of the PPARγ agonist, when administered with the amount of the Cox-2 selective inhibitor, together provide a dosage or amount of the combination that is sufficient to constitute an effective amount of the combination. The effective amount can be a cancer suppressing treatment or prevention effective amount.

In another embodiment of the subject method, a subject in need of prevention or treatment of Alzheimer's disease is treated with a PPARγagonist and a cyclooxygenase-2 selective inhibitor or prodrug thereof. In one embodiment, the subject is treated with an amount of a PPARγ agonist and an amount of a Cox-2 selective inhibitor, where the amount of the PPARγ agonist, when administered with the amount of the Cox-2 selective inhibitor, together provide a dosage or amount of the combination that is sufficient to constitute an effective amount of the combination. The effective amount can be an Alzheimer's disease suppressing treatment or prevention effective amount.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies. The phrase “therapeutically-effective” is to be understood to be equivalent to the phrase “effective for the treatment, prevention, or inhibition”, and both are intended to qualify the amount of each agent for use in the combination therapy which will achieve the goal of improvement in the severity of cancer, Alzheimer's disease, cardiovascular disease, or pain and inflammation and the frequency of incidence over treatment of each agent by itself, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

In the present method, the amount of the PPARγ agonist that is used is such that, when administered with the cyclooxygenase-2 selective inhibitor, it is sufficient to constitute an effective amount of the combination. It is preferred that the dosage of the combination constitute a therapeutically effective amount.

It is preferred that the amount of the PPARγ agonist that is used in combination with a Cox-2 selective inhibitor for a single dosage of treatment is within a range of from about 0.001 mg/kg of body weight of the subject to about 200 mg/kg. It is more preferred that the amount is from about 0.01 mg/kg to about 20 mg/kg, even more preferred that it is from about 0.1 mg/kg to about 12 mg/kg, and yet more preferred that it is from about 0.2 mg/kg to about 10 mg/kg.

The frequency of dose will depend upon the half-life of the PPARγ agonist molecule. If the PPARγ agonist molecule has a short half life (e.g. from about 2 to 10 hours) it may be necessary to give one or more doses per day. Alternatively, if the PPARγ agonist molecule has a long half-life (e.g. from about 2 to about 15 days) it may only be necessary to give a dosage once per day, per week, or even once every 1 or 2 months. A preferred dosage rate is to administer the dosage amounts described above to a subject once per day.

For the purposes of calculating and expressing a dosage rate, all dosages that are expressed herein are calculated on an average amount-per-day basis irrespective of the dosage rate. For example, one 100 mg dosage of an ingredient taken once every two days would be expressed as a dosage rate of 50 mg/day. Similarly, the dosage rate of an ingredient where 50 mg is taken twice per day would be expressed as a dosage rate of 100 mg/day.

For the purposes of calculation of a dosage rate for the present method, the weight of an adult human is assumed to be 70 kg.

The amount of Cox-2 selective inhibitor that is used in the subject method may be an amount that, when administered with the PPARγ agonist, is sufficient to constitute an effective amount of the combination. Preferably, such amount would be sufficient to provide a therapeutically effective amount of the combination. The therapeutically effective amount can also be described herein as a pain or inflammation suppressing treatment or prevention effective amount of the combination, or as a cardiovascular disorder or disease suppressing treatment or prevention effective amount, or as a cancer suppressing treatment or prevention effective amount, or as an Alzheimer's disease suppressing treatment or prevention effective amount.

In the present method, the amount of Cox-2 selective inhibitor that is used in the novel method of treatment preferably ranges from about 0.01 to about 100 milligrams per day per kilogram of body weight of the subject (mg/day.kg), more preferably from about 0.1 to about 50 mg/day.kg, even more preferably from about 1 to about 20 mg/day.kg.

When the Cox-2 selective inhibitor comprises rofecoxib, it is preferred that the amount used is within a range of from about 0.15 to about 1.0 mg/day.kg, and even more preferably from about 0.18 to about 0.4 mg/day.kg.

When the Cox-2 selective inhibitor comprises etoricoxib, it is preferred that the amount used is within a range of from about 0.5 to about 5 mg/day.kg, and even more preferably from about 0.8 to about 4 mg/day.kg.

When the Cox-2 selective inhibitor comprises celecoxib, it is preferred that the amount used is within a range of from about 1 to about 10 mg/day.kg, even more preferably from about 1.4 to about 8.6 mg/day.kg, and yet more preferably from about 2 to about 3 mg/day.kg.

When the Cox-2 selective inhibitor comprises parecoxib sodium or valdecoxib, it is preferred that the amount used is within a range of from about 0.1 to about 3 mg/day.kg, and even more preferably from about 0.3 to about 1 mg/day.kg.

In the present method, and in the subject compositions, the PPARγ agonist is administered with, or is combined with, a Cox-2 selective inhibitor. It is preferred that the weight ratio of the amount of PPARγ agonist to the amount of Cox-2 selective inhibitor that is administered to the subject is within a range of from about 0.0001:1 to about 2000:1, more preferred is a range of from about 0.002:1 to about 1200:1, even more preferred is a range of from about 0.01:1 to about 1:1.

The combination of a PPARγ agonist and a Cox-2 selective inhibitor can be supplied in the form of a novel therapeutic composition that is believed to be within the scope of the present invention. The relative amounts of each component in the therapeutic composition may be varied and may be as described just above. The PPARγ agonist and Cox-2 selective inhibitor that are described above can be provided in the therapeutic composition so that the preferred amounts of each of the components are supplied by a single dosage, a single injection or a single capsule for example, or, by up to four, or more, single dosage forms.

When the novel combination is supplied along with a pharmaceutically acceptable carrier, a pharmaceutical composition is formed. A pharmaceutical composition of the present invention is directed to a composition suitable for the prevention or treatment of pain, inflammation and/or an inflammation-associated disorder, or for the prevention or treatment of a cardiovascular disease or disorder, or for the prevention or treatment of cancer, or for the prevention or treatment of Alzheimer's disease. The pharmaceutical composition comprises a pharmaceutically acceptable carrier, a PPARγ agonist, and a cyclooxygenase-2 selective inhibitor.

Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali met al salts, alkaline earth met al salts and other physiological acceptable met al ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers and the pharmaceutically-acceptable salts of PPARγ agonists and cyclooxygenase-2 selective inhibitors. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali met al (group Ia) salts, alkaline earth met al (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The method and combination of the present invention are useful for, but not limited to, the prevention, inhibition, and treatment of pain and/or inflammation in a subject, and for treatment of inflammation-associated disorders, such as for use as an analgesic in the treatment of pain and headaches, or as an antipyretic for the treatment of fever. For example, combinations of the invention would be useful to treat arthritis, including, but not limited to, rheumatoid arthritis, spondyloarthopathies, gouty arthritis, osteoarthritis, systemic lupus erythematosus and juvenile arthritis. Such combinations of the invention would be useful in the treatment of asthma, bronchitis, menstrual cramps, tendinitis, bursitis, connective tissue injuries or disorders, and skin related conditions such as psoriasis, eczema, burns and dermatitis.

Combinations of the invention also would be useful to treat gastrointestinal conditions such as inflammatory bowel disease, gastric ulcer, gastric varices, Crohn's disease, gastritis, irritable bowel syndrome and ulcerative colitis and for the prevention or treatment of cancer, such as colorectal cancer. Combinations of the invention would be useful in treating inflammation in diseases and conditions such as herpes simplex infections, HIV, pulmonary edema, kidney stones, minor injuries, wound healing, skin wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, type II diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, and the like.

Compositions having the novel combination would also be useful in the treatment of ophthalmic diseases, such as retinitis, retinopathies, conjunctivitis, uveitis, ocular photophobia, and of acute injury to the eye tissue. The compositions would also be useful in the treatment of pulmonary inflammation, such as that associated with viral infections and cystic fibrosis. The compositions would also be useful for the treatment of certain central nervous system disorders such as cortical dementias including Alzheimer's disease. The combinations of the invention are also useful as anti-inflammatory agents, such as for the treatment of arthritis.

As used herein, the terms “pain, inflammation or inflammation-associated disorder”, and “cyclooxygenase-2 mediated disorder” are meant to include, without limitation, each of the symptoms or diseases that is mentioned above.

Several animal models are available which are appropriate for evaluation of the prevention or treatment of pain and inflammation. See, e.g., Winter et al., Proc. Soc. Exp. Biol. Med., 111:544 (1962) for the description of a rat carrageenan foot pad edema test; and Hargreaves et al., Pain 32:77 (1988), for the description of a rat carrageenan-induced analgesia test.

Animal models for arthritis are also described by Stuart, J., Ann. Rev. Immunol, 2:199 (1984). Chinn, K. S. et al., Lipids, 32(9):979-988 (1997), describe adjuvant induced arthritis by dietary arachidonic acid in essential fatty acid deficient rats.

Animal models for Alzheimer's disease are described in U.S. Pat. No. 6,310,048, to Kumar, where SAM P8 mice are used to test the effects of agents upon the synthesis of beta-amyloid protein and upon the severity of symptoms similar to those that present with Alzheimer's disease.

The present method includes the treatment and/or prevention of a cyclooxygenase-2 mediated disorder in a subject, where the method comprises treating the subject having or susceptible to the disorder with a therapeutically-effective amount of a combination of a PPARγ agonist and a compound or salt of any of the cyclooxygenase-2 selective inhibitors that are described in this specification. This method is particularly useful where the cyclooxygenase-2 mediated disorder is inflammation, arthritis, pain, or fever.

The methods and compositions described herein as the subject methods and compositions would be useful for the prevention, treatment or inhibition of cancer. Preferably, the subject methods and compositions of the present invention may be used for the treatment, prevention or inhibition of neoplasia disorders including benign and malignant neoplasias, and neoplasias in metastasis, and also including acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, breast cancer, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

Several animal models are available which are appropriate for evaluation of the prevention or treatment of cancer. For example, Petrik, M. B. et al., J. Nutr., 130(10):2434-2443 (2000) describe the use of Apc(Min/+) mice as models for testing for intestinal tumorigenesis. Desaulniers, D., et al., Environ Health Perspect, Jul: 109 (2001) describe the use of rats having mammary tumors initated by methylnitrosourea (MNU) as test subjects. Moser, A. R., et al., Cancer Tes. 61(8):3480-3485 (2001) describes the use of Apc(min)/+ mice having mammary tumors initiated by ethyinitrosourea (ENU) as model test animals.

The compositions and methods described herein would be useful for, but not limited to, the prevention, treatment or inhibition of cardiovascular disease or disorder in a subject in need of such prevention, treatment or inhibition. Such diseases and disorders may also be referred to herein as “cardiovascular/metabolic diseases and disorders” or “CVMDs”. Preferably, the compositions and methods described herein would be useful for the prevention, treatment or inhibition of inflammation-related cardiovascular disorders in a subject in need of such prevention, treatment or inhibition. The compositions and methods would be useful for prevention of coronary artery disease, aneurysm, arteriosclerosis, atherosclerosis including cardiac transplant atherosclerosis, myocardial infarction, embolism, stroke, thrombosis, including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Several animal models are available which are appropriate for evaluation of prevention of cardiovascular conditions including the prevention of atherosclerosis. See, e.g., Stehbens, Prog. Card. Dis., XXIX, 1007-28 (1986), and Zhang et al., Science, 258-468-71 (1992).

An ApoE mouse model for atherosclerosis has been described by Roselear et al. (Arterioscle. Thromb. Vasc. Biol., 16, 1013-18 (1996)). The cyclooxygenasse-2 inhibitor should be active, at a dose of 20 mg/kg, in preventing atherosclerotic lesions. Hasty, A. H., et al., J. Biol. Chem., 276(40):37402-37408 (2001), describe the use of doubly mutant mice (LDLR−/−;ob/ob) as test models for hpercholesterolemia, hypertriglyceridemia, and atherosclerosis.

As described above, an embodiment of the present invention comprises a pharmaceutical composition for the prevention of cardiovascular disorders, comprising a therapeutically-effective amount of a combination of a PPARγ agonist and a cyclooxygenase-2 selective inhibitor in association with at least one pharmaceutically-acceptable carrier, adjuvant or diluent and, if desired, other active ingredients. There are large numbers of cardiovascular treatment agents available in commercial use, in clinical evaluation and in pre-clinical development, which could be selected for use with the subject combination for the prevention of cardiovascular disorders by combination drug therapy. Such agent can be one or more agents selected from, but not limited to several major categories, namely, a lipid-lowering drug, including an IBAT inhibitor, a fibrate, niacin, a statin, a CETP inhibitor, and a bile acid sequestrant, an anti-oxidant, including vitamin E and probucol, a IIbIIIa antagonist (including xemilofiban and orbofiban), an aldosterone inhibitor (including spirolactone and epoxymexrenone), an AII antagonist (including losartan), a β-blocker, aspirin, a loop diuretic and an ace inhibitor.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of cancer, cardiovascular disease or disorder, or pain and/or inflammation associated with, but not limited to, any of the diseases or disorders described herein. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has cancer, cardiovascular disease, or pain, inflammation and/or any one of the known inflammation-associated disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

For methods of prevention, the subject is any human or animal subject, and preferably is a subject that is in need of prevention and/or treatment of cancer, cardiovascular disease, or pain, inflammation and/or an inflammation-associated disorder. The subject may be a human subject who is at risk for cancer, cardiovascular disease, or pain and/or inflammation, or for obtaining an inflammation-associated disorder, such as those described above. The subject may be at risk due to genetic predisposition, sedentary lifestyle, diet, exposure to disorder-causing agents, exposure to pathogenic agents and the like.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrases “combination therapy”, “co-administration”, “administration with”, or “co-therapy”, in defining the use of a cyclooxygenase-2 inhibitor agent and a PPARγ agonist, is intended to embrace administration of each agent in a sequential manner in a regimen that will provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

Although the combination of the present invention may include administration of a PPARγ agonist component and a cyclooxygenase-2 selective inhibitor component within an effective time of each respective component, it is preferable to administer both respective components contemporaneously, and more preferable to administer both respective components in a single delivery dose.

In particular, the combinations of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject combinations can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject combination can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The present invention further comprises kits that are suitable for use in performing the methods of treatment, prevention or inhibition described above. In one embodiment, the kit contains a first dosage form comprising a PPARγ agonist in one or more of the forms identified above and a second dosage form comprising one or more of the cyclooxygenase-2 selective inhibitors or prodrugs thereof identified above, in quantities sufficient to carry out the methods of the present invention. Preferably, the first dosage form and the second dosage form together comprise a therapeutically effective amount of the compounds for the treatment, prevention, or inhibition of pain, inflammation or inflammation-associated disorder, or of cardiovascular disease or disorder, or of cancer.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### COMPARATIVE EXAMPLE 1

This example shows the preparation of celecoxib.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇H₁₄ N₃ O₂ SF₃; C, 53.54; H, 3.70; N, 11.02. The composition that was found by analysis was: C, 53.17;H, 3.81; N, 10.90.

### EXAMPLE 2

This illustrates the production of a composition containing celecoxib and pioglitazone, and of a pharmaceutical composition containing the combination.

Pioglitazone is available in the form of pioglitazone hydrochloride under the trade name ACTOS® from Eli Lilly and Co., Indianapolis, Ind. Celecoxib can be prepared as described in Comparative Example 1, or it can be obtained under the trade name CELEBREX® from Pharmacia Corporation, Peapack, N.J.

A therapeutic composition of the present invention can be formed by intermixing pioglitazone (30 g, available as ACTOS®, from Ely Lilly and Co., Indianapolis, Ind.), and 4-[5-(4-methylphenyl)-3-(trifluoromethyl)-1H-pyrazol-1-yl]benzenesulfonamide (200 g, as produced in Comparative Example 1, or as available from Pharmacia Corporation, Peapack, N.J.), in a laboratory mill or mixing device suitable for intimate mixing of powders without substantial generation of shear or temperature sufficient to degrade either of the two compounds. After mixing, the combination of celecoxib and pioglitazone form a therapeutic composition that is sufficient for the production of about 1000 human single dose units. Each single dose unit contains about 30 mg of pioglitazone and about 200 mg of celecoxib.

If desirable, a solid carrier and other materials may be intermixed with the therapeutic composition to form a pharmaceutical composition and the resulting pharmaceutical composition may be formed into capsules for human consumption, for example, by conventional capsule-forming equipment, where each capsule contains 30 mg of pioglitazone and 200 mg celecoxib.

Alternatively, the pioglitazone and the celecoxib may be dissolved into a liquid carrier, such as, for example, normal saline solution, to form a pharmaceutical composition suitable for human consumption. A single dosage of the liquid pharmaceutical composition for human use would be a volume sufficient to provide 30 mg of pioglitazone and 200 mg of celecoxib.

Therapeutic and pharmaceutical compositions comprising a combination of any of the cyclooxygenase-2 selective inhibitors and any of the sources of PPARγ agonists that are described above can be formed by similar methods.

### EXAMPLE 3

This illustrates the evaluation of the biological efficacy of a therapeutic composition of pioglitazone and celecoxib for the alleviation of pain and inflammation.

A therapeutic composition containing pioglitazone and celecoxib is prepared as described in Example 2. The biological efficacy of the composition is determined by a rat carrageenan foot pad edema test and by a rat carrageenan-induced analgesia test.

Rat Carrageenan Foot Pad Edema Test:

The carrageenan foot edema test is performed with materials, reagents and procedures essentially as described by Winter, et al., (Proc. Soc. Exp. Biol. Med., 111, 544 (1962)). Male Sprague-Dawley rats are selected in each group so that the average body weight is as close as possible. Rats are fasted with free access to water for over sixteen hours prior to the test. The rats are dosed orally (1 mL) with compounds of Example 2 suspended in a carrier vehicle containing 0.5% methylcellulose and 0.025% surfactant, or with only the carrier vehicle alone. One hour later, a subplantar injection of 0.1 mL of 1% solution of carrageenan/sterile 0.9% saline is administered to one foot and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDS, in Non-steroidal Anti-Inflammatory Drugs, (J. Lombardino, ed. 1985)). The percent inhibition shows the percent decrease from control paw volume determined in this procedure. It is believed that the data would show that the combination of pioglitazone and celecoxib provides effective anti-inflammatory activity.

Rat Carrageenan-Induced Analgesia Test:

The analgesia test using rat carrageenan is performed with materials, reagents and procedures essentially as described by Hargreaves, et al., (Pain, 32, 77 (1988)). Male Sprague-Dawley rats are treated as previously described for the Carrageenan Foot Pad Edema test. Three hours after the injection of the carrageenan, the rats are placed in a special PLEXIGLAS® container with a transparent floor having a high intensity lamp as a radiant heat source, positionable under the floor. After an initial twenty-minute period, thermal stimulation is begun on either the injected foot or on the contralateral uninjected foot. A photoelectric cell will turn off the lamp and timer when the light is interrupted by paw withdrawal. The time until the rat withdraws its foot is then measured. The withdrawal latency in seconds is determined for the control and drug-treated groups, and percent inhibition of the hyperalgesic foot withdrawal is determined. It is believed that results would show that a combination of pioglitazone and celecoxib provides effective analgesic activity.

### EXAMPLE 4

This illustrates the biological efficacy of a therapeutic composition of pioglitazone and celecoxib for the treatment of collagen-induced arthritis in mice.

A therapeutic composition containing pioglitazone and celecoxib is prepared as described in Example 2. The biological efficacy of the composition is determined by induction and assessment of collagen-induced arthritis in mice.

Arthritis is induced in 8-12 week old male DBA/1 mice by injection of 50 μg of chick-type II collagen (CII) in complete Freunds adjuvant (Sigma) on day 0 at the base of the tail as described in [J. Stuart, Annual Rev. Immunol., 2, 199 (1984)]. Compounds are prepared as a suspension in 0.5% methylcellulose (Sigma, St. Louis, Mo.), and 0.025% Tween 20 (Sigma). The cyclooxygenase-2 inhibitor (celecoxib, as described in Comparative Example 1), and pioglitazone (available as pioglitazone hydrochloride under the trade name ACTOS® from Ely Lilly and Company, Indianapolis, Ind.) are administered alone or in combination as a therapeutic composition as described in Example 2. The compounds are administered in non-arthritic animals by gavage in a volume of 0.1 ml beginning on day 20 post collagen injection and continuing daily until final evaluation on day 55. Animals are boosted on day 21 with 50 μg of collagen (CII) in incomplete Freunds adjuvant. The animals are subsequently evaluated several times each week for incidence and severity of arthritis until day 56. Any animal with paw redness or swelling is counted as arthritic. Scoring of severity is carried out using a score of 0-3 for each paw (maximal score of 12/mouse) as described in P. Wooley, et al., Trans. Proc., 15, 180 (1983). The animals are measured for incidence of arthritis and severity in the animals where arthritis was observed. The incidence of arthritis is determined at a gross level by observing the swelling or redness in the paw or digits. Severity is measured with the following guidelines. Briefly, animals displaying four normal paws, i.e., no redness or swelling are scored 0. Any redness or swelling of digits or the paw are scored as 1. Gross swelling of the whole paw or deformity is scored as 2. Ankylosis of joints is scored as 3.

Histological Examination of Paws:

In order to verify the gross determination of a non-arthritic animal, a histological examination can be performed. Paws from animals sacrificed at the end of the experiment are removed, fixed and decalcified as previously described [R. Jonsson, J. Immunol. Methods, 88, 109 (1986)]. Samples are paraffin embedded, sectioned, and stained with hematoxylin and eosin by standard methods. Stained sections are examined for cellular infiltrates, synovial hyperplasia, and bone and cartilage erosion.

It is believed that results will show that the combination of a cyclooxygenase-2 selective inhibitor with the PPARγ agonist pioglitazone was an efficacious treatment for collagen-induced arthritis in mice.

It is believed that Examples 3 and 4 can be repeated with compositions comprising any of the PPARγ agonists in combination with any of the cyclooxygenase-2 selective inhibitors that are described herein, with the results showing that the combination provides effective anti-inflammatory activity, effective analgesic activity, and is an efficacious treatment of collagen-induced arthritis in mice.

### EXAMPLE 5

This example illustrates the efficacy of a PPARγ agonist in combination with a cyclooxygenase-2 selective inhibitor for the treatment of cancer.

A combination of any one or more of the PPARγ agonists that are described herein with any one or more of the cyclooxygenase-2 selective inhibitors that are described herein can be prepared by the methods described in Example 2. The efficacy of the combination can be tested by the methods described in U.S. Pat. No. 6,242,196, for:

a. the reduction in size of adipose cell tumors in vivo;

b. the inhibition of proliferation of leukemic cells; and

c. the inhibition of proliferation of prostate cancer cells.

It is believed that the subject combinations would be found to be effective in reducing the size of adipose cell tumors in vivo; in inhibiting the proliferation of leukemic cells; and in inhibiting the proliferation of prostate cancer cells.

### EXAMPLE 6

This example illustrates the efficacy of a PPARγ agonist in combination with a cyclooxygenase-2 selective inhibitor for the improvement of cardiac function in myocardial infarction.

A combination of any one or more of the PPARγ agonists that are described herein with any one or more of the cyclooxygenase-2 selective inhibitors that are described herein can be prepared by the methods described in Example 2. The efficacy of the combination can be tested by the methods described by Saito, T. et al., in Biochem. and Biophys. Res. Communic., 273:772-775 (2000), for the improvement of cardiac function in myocardial infarction. It is believed that the subject combinations would be found to be effective in improving cardiac function in myocardial infarction.

### EXAMPLE 7

This example illustrates the efficacy of a combination of celecoxib and pioglitazone in alleviating adjuvant induced arthritis in rats.

A combination of celecoxib and pioglitazone can be prepared by the methods described in Example 2. The efficacy of the combination can be tested by the method described by Chinn, K. S. et al., in Lipids, 32(9):979-988 (1997).

It is believed that the subject combination would be found to be effective in alleviating adjuvant induced arthritis in rats. In fact, it is believed that a combination that included any one or more of the PPARγ agonists that are described herein with any one or more of the cyclooxygenase-2 selective inhibitors that are described herein would also be effective for such purpose.

### EXAMPLE 8

This example illustrates the efficacy of a combination of celecoxib and pioglitazone in preventing or treating intestinal tumors in Apc (Min/+) mice.

A combination of celecoxib and pioglitazone can be prepared by the methods described in Example 2. The efficacy of the combination in preventing or reducing intestinal tumorigenesis in Apc (Min/+) mice can be tested by the method described by Petrik, M. B. H. et al., in J. Nutr., 130:2434-2443 (2000).

It is believed that the subject combination would be found to be effective in preventing or reducing tumoregenesis in such mice. In fact, it is believed that a combination that included any one or more of the PPARγ agonists that are described herein with any one or more of the cyclooxygenase-2 selective inhibitors that are described herein would also be effective for such purpose.

### EXAMPLE 9

This example illustrates the efficacy of a combination of celecoxib and pioglitazone in preventing or treating mammary hyperplasias and carcinomas in Apc(min/+) mice.

A combination of celecoxib and pioglitazone can be prepared by the methods described in Example 2. The efficacy of the combination for the prevention or treatment of mammary hyperplasias and carcinomas in mice can be tested by the method described by Moser, A. R. et al., Cancer Res., 61(8):3480-3485 (2001), (for cancers induced by ethylnitrosourea (ENU)), or in rats by the method described by Deasulniers, D. et al., Environ. Health Perspect., 109(7):739-747 (2001), (for cancers induced by methylnitrosourea (MNU)).

It is believed that the subject combination would be found to be effective in prevention or treating mammary tumor development in mice and rats. In fact, it is believed that a combination that included any one or more of the PPARγ agonists that are described herein with any one or more of the cyclooxygenase-2 selective inhibitors that are described herein would also be effective for such purpose.

### EXAMPLE 10

This example illustrates the efficacy of a combination of celecoxib and pioglitazone in preventing or treating hypercholesterolemia, hypertriglyceridemia and atherosclerosis in mice.

A combination of celecoxib and pioglitazone can be prepared by the methods described in Example 2. The efficacy of the combination for the prevention or treatment of hypercholesterolemia, hypertriglyceridemia and atherosclerosis in mice can be tested by the method described by Hasty, A. H. et al., J. Biol. Chem., 276(40):37402-37408 (2001). The method uses doubly mutant LDLR−/−;ob/ob mice as the model animal.

It is believed that the subject combination would be found to be effective in preventing and/or treating hypercholesterolemia, hypertriglyceridemia and atherosclerosis in mice. In fact, it is believed that a combination that included any one or more of the PPARγ agonists that are described herein with any one or more of the cyclooxygenase-2 selective inhibitors that are described herein would also be effective for such purpose.

### EXAMPLE 11

This example illustrates the efficacy of a combination of celecoxib and pioglitazone in reducing cardiovascular risk in humans.

A combination of celecoxib and pioglitazone can be prepared by the methods described in Example 2. The efficacy of the combination can be tested by the methods described in any one of the references cited in Table 1, of the publication by Robins, S. J., in J. Cardiovascular Risk, 8:195-201 (2001).

It is believed that the subject combination would be found to be effective in reducing cardiovascular risk in humans. In fact, it is believed that a combination that included any one or more of the PPARγ agonists that are described herein with any one or more of the cyclooxygenase-2 selective inhibitors that are described herein would also be effective for such purpose.

### EXAMPLE 12

This example illustrates the efficacy of a combination of celecoxib and pioglitazone in preventing or treating diabetes in rats.

A combination of celecoxib and pioglitazone can be prepared by the methods described in Example 2. The efficacy of the combination for the prevention or treatment of type 2 diabetes in Zucker diabetic fatty rats (ZDF) can be tested by the method described by Shibata, T. et al., in Br. J. Pharmacol., 130(3):495-504 (2000).

It is believed that the subject combination would be found to be effective in preventing and/or treating type 2 diabetes in rats. In fact, it is believed that a combination that included any one or more of the PPARγ agonists that are described herein with any one or more of the cyclooxygenase-2 selective inhibitors that are described herein would also be effective for such purpose.

### EXAMPLE 13

This example illustrates the efficacy of a combination of celecoxib and pioglitazone in preventing or treating Alzheimer's disease in mice.

A combination of celecoxib and pioglitazone can be prepared by the methods described in Example 2. The efficacy of the combination can be tested for the ability to prevent or treat the production and accumulation of amyloid beta protein and for the ability to prevent or alleviate Alzheimer's disease-type symptoms in SAM P8 mice by the method described in U.S. Pat. No. 6,310,048 to Kumar.

It is believed that the subject combination would be found to be effective in preventing and/or treating Alzheimer's disease in mice. In fact, it is believed that a combination that included any one or more of the PPARγ agonists that are described herein with any one or more of the cyclooxygenase-2 selective inhibitors that are described herein would also be effective for such purpose. Alzheimer's disease, U.S. Pat. No. 6,310,048 to Kumar

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A method for the prevention, treatment, or inhibition of pain, inflammation, or inflammation-related disorder, or cancer, or Alzheimer's disease, or cardiovascular disease or disorder in a subject in need of such treatment, prevention, or inhibition, the method comprising treating the subject with a peroxisome proliferator activated receptor-γ agonist and a cyclooxygenase-2 selective inhibitor or prodrug thereof.

2. The method according to claim 1, wherein the method is for the treatment of pain, inflammation, or inflammation-related disorder in a subject in need of such treatment, prevention, or inhibition.

3. The method according to claim 1, wherein the peroxisome proliferator activated receptor-γ agonist comprises a material that is selected from the group consisting of thiazolidinediones, non-steroidal anti-inflammatory drugs which are capable of binding with PPARγ, indomethacin, flufenamic acid, fenoprofen, ibuprofen, unsaturated fatty acids which are capable of binding with PPARγ; prostaglandins which are capable of binding with PPARγ, prostaglandin J₂ analogs which are capable of binding with PPARγ, and mixtures thereof.

4. The method according to claim 3, wherein the peroxisome proliferator-activated receptor-γ agonist comprises a thiazolidinedione.

5. The method according to claim 4, wherein the peroxisome proliferator-activated receptor-γ agonist comprises a compound that is selected from the group consisting of CS-011, AD-5075, BRL-49653, AY-31637, MCC-555, ciglitazone, darglitazone, englitazone, pioglitazone, rosiglitazone, trogliltazone, 5-[[4-[2-(methyl-2-pyridinylamino)ethoxy]phenyl]methyl]-2,4-thiazolidinedione, and mixtures thereof.

6. The method according to claim 1, wherein the peroxisome proliferator-activated receptor-γ agonist comprises a compound that is selected from the group consisting of GW1929, JTT501, PD72953, WAY-120,744, L-764406, GG520, indomethacin, (−)3-[4-[2-(phenoxazin-10-yl)ethoxy]phenyl]-2-ethoxypropanoic acid, and mixtures thereof.

7. The method according to claim 1, wherein the peroxisome proliferator-activated receptor-γ agonist comprises docosahexanoic acid, prostaglandin J₂, or an analog of prostaglandin J₂.

8. The method according to claim 7, wherein the analog of prostaglandin J₂ comprises Δ¹²-prostaglandin J₂, or 15-deoxy-Δ^{12,14}-prostaglandin J₂.

9. The method according to claim 1, wherein the peroxisome poroliferator-activated receptor-γ comprises a compound are having the structure: 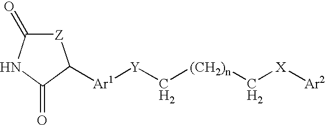
wherein
Ar¹ is (1) arylene or
(2) heteroarylene,
wherein arylene and heteroarylene are optionally substituted with from 1 to 4 groups selected from Rₐ;
Ar² is (1) ortho-substituted aryl or
(2) ortho-substituted heteroaryl,
wherein said ortho substituent is selected from R; and aryl and heteroaryl are optionally further substituted with from 1-4 groups independently selected from R^{a};
X and Y are independently O, S, N—R^{b}, or CH₂;
Z is O or S;
n is 0 to 3;
R is (1) C₃₋₁₀ alkyl optionally substituted with 1-4 groups selected from halo and C₃₋₆ cycloalkyl,
(2) C₃₋₁₀ alkenyl, or
(3) C₃₋₈ cycloalkyl;
R^{a} is (1) C₁₋₅ alkanoyl,
(2) C₁₋₅ alkyl,
(3) C₂₋₁₅ alkenyl,
(4) C₂₋₁₅ alkynyl,
(5) halo,
(6) OR^{b},
(7) aryl, or
(8) heteroaryl,
wherein said alkyl, alkenyl, alkynyl, and alkanoyl are optionally substituted with from 1-5 groups selected from R^{c}, and said aryl and heteroaryl optionally substituted with 1 to 5 groups selected from R^{d}.
R^{b} is (1) hydrogen,
(2) C₁₋₁₀ alkyl,
(3) C₂₋₁₀ alkenyl,
(4) C₂₋₁₀ alkynyl,
(5) aryl,
(6) heteroaryl,
(7) aryl C₁₋₁₅ alkyl,
(8) heteroaryl C₁₋₅ alkyl,
(9) C₁₋₅ cycloalkyl,
(10) C₃₋₈ cycloalkyl,
wherein alkyl, alkenyl, alkynyl are optionally substituted with one to four substituents independently selected from R^{c}, and cycloalkyl, aryl, and heteroaryl are optionally substituted with one to four substituents independently selected from R^{d}; or
R^{c} is (1) halo,
(2) aryl,
(3) heteroaryl,
(4) CN,
(5) NO₂,
(6) OR^{f},
(7) S(O)ₘR^{f}, m=0, 1 or 2, provided that R^{f} is not H when m is 1 or 2;
(8) NR^{f}R^{f},
(9) NR^{f}COR^{f},
(10) NR^{f}CO₂R^{f},
(11) NR^{f}CON(R^{f})₂,
(12) NR^{f}SO₂R^{f}, provided that R^{f} is not H,
(13) COR^{f},
(14) CO₂R^{f},
(15) CON(R^{f})₂,
(16) SO₂N(R^{f})₂,
(17) OCON(R^{f})₂, or
(18) C₃₋₈ cycloalkyl,
wherein said cycloalkyl, aryl and heteroaryl are optionally substituted with 1 to 3 groups of halo or C₁₋₆ alkyl;
R^{d} is (1) a group selected from R^{c},
(2) C₁₋₁₀ alkyl,
(3) C₂₋₁₀ alkenyl,
(3) C₂₋₁₀ alkenyl,
(4) C₂₋₁₀ alkynyl,
(5) aryl C₁₋₁₀ alkyl, or
(6) heteroaryl C₁₋₁₀ alkyl,
wherein alkyl, alkenyl, alkynyl, aryl, heteroaryl are optionally substituted with a group independently selected from R^{e};
R^{e} is (1) halogen,
(2) amino,
(3) carboxyl,
(4) C₁₋₄ alkyl,
(5) C₁₋₄ alkoxy,
(6) hydroxy,
(7) aryl,
(8) aryl C₁₋₄ alkyl, or
(9) aryloxy;
R^{f} is (1) hydrogen,
(2) C₁₋₁₀ alkyl,
(3) C₂₋₁₀ alkenyl,
(4) C₂₋₁₀ alkynyl,
(5) aryl,
(6) heteroaryl,
(7) aryl C₁₋₁₅ alkyl,
(8) heteroaryl C₁₋₁₅alkyl,
(9) C₁₋₁₅ alkanoyl,
(10) C₃₋₈ cycloalkyl;
wherein alkyl, alkenyl, alkynyl, aryl, heteroaryl, alkanoyl and cycloalkyl are optionally substituted with one to four groups selected from R^{e};
or a pharmaceutically acceptable salt thereof.

10. The method according to claim 1, wherein the cyclooxygenase-2 selective inhibitor or prodrug thereof has a cyclooxygenase-2 IC₅₀ of less than about 0.2 μmol/L.

11. The method according to claim 10, wherein the cyclooxygenase-2 selective inhibitor or prodrug thereof has a cyclooxygenase-1 IC₅₀ of at least about 1 μmol/L.

12. The method according to claim 1, wherein the cyclooxygenase-2 selective inhibitor is selected from the group consisting of celecoxib, valdecoxib, deracoxib, rofecoxib, etoricoxib, parecoxib, lumiracoxib, SD-8381, ABT-963, BMS-347070, and NS-398.

13. The method according to claim 12, wherein the cyclooxygenase-2 selective inhibitor is selected from the group consisting of celecoxib, valdecoxib, deracoxib, rofecoxib, etoricoxib, parecoxib, and lumiracoxib.

14. The method according to claim 13, wherein the cyclooxygenase-2 selective inhibitor is selected from the group consisting of celecoxib, valdecoxib, and parecoxib.

15. The method according to claim 1, wherein the cyclooxygenase-2 selective inhibitor comprises celecoxib.

16. The method according to claim 2, wherein the amount of peroxisome proliferator activated receptor-γ agonist, together with the amount of the cyclooxygenase-2 selective inhibitor or prodrug thereof, constitute an amount effective for the treatment, prevention, or inhibition of the pain, inflammation or inflammation-associated disorder.

17. The method according to claim 1, wherein the amount of peroxisome proliferator activated receptor-γ agonist is within a range of from about 0.01 to about 20 mg/day per kg of body weight of the subject.

18. The method according to claim 17, wherein the amount of the cyclooxygenase-2 selective inhibitor or prodrug thereof is within a range of from about 0.01 to about 100 mg/day per kg of body weight of the subject.

19. The method according to claim 18, wherein the amount of the cyclooxygenase-2 selective inhibitor or prodrug thereof is within a range of from about 1 to about 20 mg/day per kg of body weight of the subject.

20. The method according to claim 1, wherein the weight ratio of the amount of peroxisome proliferator activated receptor-γ agonist to the amount of cyclooxygenase-2 selective inhibitor or prodrug thereof that is administered to the subject is within a range of from about 0.002:1 to about 1200:1.

21. The method according to claim 20, wherein the weight ratio of the amount of peroxisome proliferator activated receptor-γ agonist to the amount of cyclooxygenase-2 selective inhibitor or prodrug thereof that is administered to the subject is within a range of from about 0.01:1 to about 1:1.

22. The method according to claim 2, wherein the pain, inflammation or inflammation associated disorder is selected from the group consisting of headache, fever, arthritis, rheumatoid arthritis, spondyloarthopathies, gouty arthritis, osteoarthritis, systemic lupus erythematosus, juvenile arthritis, asthma, bronchitis, menstrual cramps, tendinitis, bursitis, connective tissue injuries or disorders, skin related conditions, psoriasis, eczema, burns, dermatitis, gastrointestinal conditions, inflammatory bowel disease, gastric ulcer, gastric varices, Crohn's disease, gastritis, irritable bowel syndrome, ulcerative colitis, cancer, colorectal cancer, herpes simplex infections, HIV, pulmonary edema, kidney stones, minor injuries, wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, myasthenia gravis, multiple sclerosis, sarcoidosis, nephrotic syndrome, Behcet's syndrome, polymyositis, gingivitis, hypersensitivity, swelling occurring after injury, myocardial ischemia, ophthalmic diseases, retinitis, retinopathies, conjunctivitis, uveitis, ocular photophobia, acute injury to the eye tissue, pulmonary inflammation, nervous system disorders, cortical dementias, and Alzheimer's disease.

23. The method according to claim 2, wherein the pain, inflammation or inflammation associated disorder is an opthalmic disease or opthalmic injury.

24. The method according to claim 23, wherein the opthalmic disease or opthalmic injury is selected from the group consisting of retinitis, retinopathies, conjunctivitis, uveitis, ocular photophobia, acute injury to the eye tissue.

25. The method according to claim 22, wherein the pain, inflammation or inflammation associated disorder is arthritis.

26. The method according to claim 25, wherein the arthritis is osteoarthritis.

27. The method according to claim 25, wherein the arthritis is rheumatoid arthritis.

28. The method according to claim 1, wherein the subject is an animal.

29. The method according to claim 28, wherein the subject is a human.

30. The method according to claim 1, wherein the treating step comprises administering a peroxisome proliferator activated receptor-γ agonist and a cycloxoygenase-2 selective inhibitor to the subject enterally or parenterally in one or more dose per day.

31. The method according to claim 30, wherein the peroxisome proliferator activated receptor-γ agonist and the cycoloxygenase-2 selective inhibitor are administered to the subject substantially simultaneously.

32. The method according to claim 30, wherein the peroxisome proliferator activated receptor-γ agonist and the cycoloxygenase-2 selective inhibitor are administered sequentially.

33. A method for the treatment or prevention of disorders having an inflammatory component in a subject in need of the treatment or prevention of disorders having an inflammatory component, the method comprising administering to the subject a therapeutically effective dose of a peroxisome proliferator activated receptor-γ agonist and a cyclooxygenase-2 selective inhibitor or a pharmaceutically acceptable salt or prodrug thereof.

34. A composition for the treatment, prevention, or inhibition or pain, inflammation, or inflammation-associated disorder comprising a peroxisome proliferator activated receptor-γ agonist and a cyclooxygenase-2 selective inhibitor or prodrug thereof.

35. The composition according to claim 34, wherein the composition is useful for treating a subject in need of treatment, prevention, or inhibition of pain, inflammation, or an inflammation-associated disorder, and wherein a dose of the composition constitutes an amount of peroxisome proliferator activated receptor-γ agonist and an amount of a cyclooxygenase-2 selective inhibitor or a pharmaceutically acceptable salt or prodrug thereof which together constitute a pain or inflammation suppressing treatment or prevention effective amount.

36. A pharmaceutical composition comprising a peroxisome proliferator activated receptor-γ agonist; a cyclooxygenase-2 selective inhibitor or prodrug thereof; and a pharmaceutically-acceptable excipient.

37. A kit that is suitable for use in the treatment, prevention or inhibition of pain, inflammation or inflammation-associated disorder, the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the combination of the compounds for the treatment, prevention, or inhibition of pain, inflammation or inflammation-associated disorder.

38. A method for the treatment, prevention, or inhibition of cardiovascular disease or disorder in a subject in need of such treatment, prevention, or inhibition, the method comprising treating the subject with a peroxisome proliferator-activated receptor-γ agonist and a cyclooxygenase-2 selective inhibitor or a pharmaceutically acceptable salt or prodrug thereof.

39. The method according to claim 38, wherein the cyclooxygenase-2 selective inhibitor is selected from the group consisting of celecoxib, valdecoxib, deracoxib, rofecoxib, etoricoxib, parecoxib, lumiracoxib, SD-8381, ABT-963, BMS-347070, and NS-398.

40. The method according to claim 39, wherein the cyclooxygenase-2 selective inhibitor is selected from the group consisting of celecoxib, valdecoxib, deracoxib, rofecoxib, etoricoxib, parecoxib, and lumiracoxib.

41. The method according to claim 40, wherein the cyclooxygenase-2 selective inhibitor is selected from the group consisting of celecoxib, valdecoxib, and parecoxib.

42. The method according to claim 38, wherein the cyclooxygenase-2 selective inhibitor comprises celecoxib.

43. The method according to claim 38, wherein the cardiovascular disease or disorder is selected from the group consisting of coronary artery disease, aneurysm, arteriosclerosis, atherosclerosis, cardiac transplant atherosclerosis, myocardial infarction, embolism, stroke, thrombosis, venous thrombosis, angina including unstable angina, coronary plaque inflammation, bacterial-induced inflammation, Chlamydia-induced inflammation, viral induced inflammation, inflammation associated with surgical procedures, vascular grafting, coronary artery bypass surgery, revascularization procedures, angioplasty, stent placement, endarterectomy, and inflammation associated with other invasive procedures involving arteries, veins and capillaries.

44. A composition for the treatment, prevention, or inhibition of cardiovascular disease or disorder comprising a peroxisome proliferator activated receptor-γ agonist and a cyclooxygenase-2 selective inhibitor or prodrug thereof.

45. A kit that is suitable for use in the treatment, prevention, or inhibition of cardiovascular disease or disorder, wherein the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the compounds for the treatment, prevention, or inhibition of cardiovascular disease or disorder.

46. A method for the treatment, prevention, or inhibition of cancer in a subject in need of such treatment, prevention, or inhibition, the method comprising treating the subject with a peroxisome proliferator-activated receptor-γ agonist and a cyclooxygenase-2 selective inhibitor or a pharmaceutically acceptable salt or prodrug thereof.

47. The method according to claim 46, wherein the cyclooxygenase-2 selective inhibitor is selected from the group consisting of celecoxib, valdecoxib, deracoxib, rofecoxib, etoricoxib, parecoxib, lumiracoxib, SD-8381, ABT-963, BMS-347070, and NS-398.

48. The method according to claim 47, wherein the cyclooxygenase-2 selective inhibitor is selected from the group consisting of celecoxib, valdecoxib, deracoxib, rofecoxib, etoricoxib, parecoxib, and lumiracoxib.

49. The method according to claim 48, wherein the cyclooxygenase-2 selective inhibitor is selected from the group consisting of celecoxib, valdecoxib, and parecoxib.

50. The method according to claim 46, wherein the cyclooxygenase-2 selective inhibitor comprises celecoxib.

51. The method according to claim 46, wherein the cancer is selected from the group consisting of neoplasia disorders, benign neoplasias, neoplasias in metastasis, malignant neoplasias, acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, breast cancers, colon cancers, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

52. A composition for the treatment, prevention, or inhibition of cancer comprising a peroxisome proliferator activated receptor-γ agonist and a cyclooxygenase-2 selective inhibitor or prodrug thereof.

53. A kit that is suitable for use in the treatment, prevention, or inhibition of cancer, wherein the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the compounds for the treatment, prevention, or inhibition of cancer.

54. A method for the treatment, prevention, or inhibition of Alzheimer's disease in a subject in need of such treatment, prevention, or inhibition, the method comprising treating the subject with a peroxisome proliferator-activated receptor-s agonist and a cyclooxygenase-2 selective inhibitor or a pharmaceutically acceptable salt or prodrug thereof.

55. A composition for the treatment, prevention, or inhibition of Alzheimer's disease comprising a peroxisome proliferator activated receptor-γ agonist and a cyclooxygenase-2 selective inhibitor or prodrug thereof.

56. A kit that is suitable for use in the treatment, prevention, or inhibition of Alzheimer's disease, the kit comprises a first dosage form comprising a peroxisome proliferator activated receptor-γ agonist and a second dosage form comprising a cyclooxygenase-2 selective inhibitor or prodrug thereof, in quantities which comprise a therapeutically effective amount of the compounds for the treatment, prevention, or inhibition of Alzheimer's disease.
